(19) United States
(12) Patent Application Publication
(10) Pub. No.: US 2022/0266242 A1
(43) Pub. Date: Aug. 25, 2022

(54) RAPID BLOOD TESTING SYSTEM

(71) Applicant: InViDx Corp., Schomberg (CA)

(72) Inventor: James Samsoondar, Schomberg (CA)

(21) Appl. No.: 17/744,283

(22) Filed: May 13, 2022

Related U.S. Application Data
(63) Continuation of application No. PCT/CA2021/051289, filed on Sep. 15, 2021.
Application No. PCT/CA2021/051289 is a continuation-in-part of application No. PCT/CA2020/051254, filed on Sep. 18, 2020.
Application No. PCT/CA2020/051254 is a continuation-in-part of application No. 16/854,201, filed on Apr. 21, 2020, now Pat. No. 11,161,109.
Application No. 16/854,201 is a continuation-in-part of application No. 16/575,645, filed on Sep. 19, 2019, now Pat. No. 11,327,084.

(30) Foreign Application Priority Data
Dec. 23, 2019 (CA) 3066133
May 19, 2020 (CA) 3081050
Sep. 18, 2020 (CA) PCT/CA2020/051254
Feb. 3, 2021 (CA) 3107994

(51) Int. Cl. B01L 3/00; G01N 35/10; G01N 33/49; G01N 27/416

(52) U.S. Cl. CPC B01L 3/5027; G01N 35/1095; G01N 33/4925; G01N 27/4167; B01L 2200/0684; B01L 2200/16; B01L 2300/045; B01L 2300/087; B01L 2400/0481; B01L 2400/0638; B01L 2400/0683

(57) ABSTRACT

A point-of-care testing (POCT) system comprising an analyzer, a measurement cartridge, and a calibration cartridge for calibrating the measurement cartridge is described. The measurement cartridge comprises at least one electrochemical sensor for measuring the one or more properties of the blood sample, and the calibration cartridge comprises a similar at least one electrochemical sensor and at least one sealed blister containing calibration fluid for calibrating the measurement cartridge. Examples of properties of the blood sample may be pH, blood gases, electrolytes, and metabolites like glucose and creatinine. The measurement cartridge may also comprise an optical chamber for measuring for example, bilirubin and hemoglobin species, for which the analyzer comprises stored calibration algorithms. The optical chamber may be disposed in any location in the measurement cartridge, for receiving a portion of the blood sample.

### CROSS-REFERENCE TO RELATED APPLICATIONS

This application is a continuation of PCT Application No. PCT/CA2021/051289 filed Sep. 15, 2021, which claims the benefit of Canadian Patent No. 3,107,994 filed Feb. 3, 2021 and PCT Application No. PCT/CA2020/051254 filed Sep. 18, 2020. PCT Application No. PCT/CA2020/051254 claims the benefit of U.S. application Ser. No. 16/854,201 filed Apr. 21, 2020, now U.S. Pat. No. 11,161,109, and claims the benefit of Canadian Application No. 3,081,050 filed May 19, 2020, which claims the benefit of U.S. application Ser. No. 16/854,201 filed Apr. 21, 2020, now U.S. Pat. No. 11,161,109, which claims the benefit of Canadian Application No. 3,066,133 filed December 2019, which claims the benefit of U.S. application Ser. No. 16/575,645 filed Sep. 19, 2019, now U.S. Pat. No. 11,327,084. U.S. application Ser. No. 16/854,201 filed Apr. 21, 2020, now U.S. Pat. No. 11,161,109, is a continuation in part of U.S. application Ser. No. 16/575,645 filed Sep. 19, 2019, now U.S. Pat. No. 11,327,084, all of which are herein incorporated by reference.

### FIELD OF THE INVENTION

The invention relates to a point-of-care testing (POCT) system comprising an analyzer and a measurement cartridge having one or more detection chambers. The detection chamber of the measurement cartridge may comprise one or more electrochemical sensors and/or one or more optical chambers. The system may also comprise a calibration cartridge for calibrating at least one of the one or more electrochemical sensors.

### BACKGROUND OF THE INVENTION

In the clinical laboratory, a tissue substance from the body that is undergoing analysis is usually referred to as an analyte or a test. “Point-of-care Testing” (POCT) is defined as medical diagnostic testing performed in close proximity to where the patient is receiving care. Point-of-care (POC) is not restricted to laboratory tests but are more common with respect to laboratory tests. POCT is usually performed by non-laboratory personnel and the results are used for clinical decision making. An example of a non-laboratory POC device is a POC ultrasound (POCUS) device.

For the sake of convenience and rapid turnaround time, the tissue or sample of choice for POCT is whole blood (also referred to as blood). Due to the complexity of blood, certain tests can only be performed on serum or plasma. Regardless whether the sample is serum, plasma or whole blood, the quantities of analytes measured are usually measured in the plasma component of whole blood and are usually reported as a mass or molar quantity per unit volume of the whole blood used for analysis. Sometimes it is preferred to lyse the red blood cells before measurement, whereby the contents of the red blood cells become mixed with the plasma. Because the actual volume of plasma present in the blood depends on the hematocrit, some systems attempt to correct the measured values to account for hematocrit. The hematocrit is the proportion, by volume, of the blood that consists of red blood cells.

When blood is allowed to clot and the sample is centrifuged, the yellow liquid that sits on top of the blood clot is called serum. If the blood is collected in a tube containing an anticoagulant, for example heparin, and the blood centrifuged, the cells and cell fragments, referred to as formed elements, are separated from a yellow liquid called plasma, which sits on top of the formed elements. The plasma is usually about 90 percent water, in which the formed elements are usually suspended, and it transports nutrients as well as wastes throughout the body. Various analytes are dissolved in the plasma for example, glucose, electrolytes, blood gases, drugs, hormones, lipids, enzymes (e.g., ALT, which may be used for assessing liver function), and metabolites (e.g., creatinine which may be used for assessing kidney function, and lactate which may be used for detecting sepsis).

POCT involves a range of procedures of varying complexity that may include manual procedures and automated procedures conducted by portable analyzers. POCT is most efficient when the sample of interest can be applied to or loaded onto a measurement cartridge or a test cartridge at a cartridge opening (may also be referred to as a sample inlet of the cartridge), capped, and the analytical or testing steps performed automatically after the capped cartridge is inserted into a slot or receptor of an associated analyzer. Some POCT require one or more reagent that reacts with the blood sample, providing altered blood. The result of reaction between a liquid sample and one or more reagent may depend on the quantity of the one or more reagent and the volume of liquid sample. The reagent is preferably in a dry form, in order to avoid dilution of the sample.

Some blood tests, for example coagulation assays and immunoassays, require a fixed volume of sample or metered volume of sample to ensure that when mixed with a reagent, the ratio of the volume of sample to the volume (or mass) of the reagent is held constant. The term metered blood means that the blood is supplied in a measured or regulated amount. In other cases, for example the measurement of blood gases and electrolytes, a metered volume of sample is not required. In the case of electrolytes, the volume of the sample is usually not an issue if the electrolyte concentration is estimated by measuring electrical activity in the sample. The term blood gases may refer to pH, pCO₂ [partial pressure of carbon dioxide] and pO₂ [partial pressure of oxygen] and the term electrolytes may refer to sodium, potassium, chloride and bicarbonate ions. Other ions like calcium ions may also be referred to as electrolytes. Electrical activity is usually measured using electrochemical sensors, also referred to as biosensors. Blood gases and electrolytes are mostly measured by electrochemical sensors, but optical measurements are also possible.

There are other tests that do not require a fixed volume of sample, and cannot be measured using biosensors, for example CO-oximetry. CO-oximetry is a spectroscopic or optical technique that is used to measure the amount of different Hemoglobin (Hb) species present in a blood sample, for example, Oxy-Hb, Deoxy-Hb, Met-Hb, Carboxy-Hb and Total-Hb, and their measurements are used to assess the oxygenation status of a patient. Met-Hb and Carboxy-Hb are non-functional hemoglobin and elevated levels can be life-threatening. Although electrolytes and CO-oximetry measurements do not usually require fixed volumes of blood, the distance the blood sample travels along microfluidic channels inside some cartridges may need to be controlled or metered.

Hemoglobin is an example of an analyte that is not present in the plasma unless hemolysis has occurred. Hemoglobin is usually present in red blood cells (RBCs), and the mass or molar concentration of hemoglobin may be measured in altered blood (may be simply hemolyzed blood) or unaltered blood. Hemolyzed blood may be produced using sound waves or chemicals. Some analyzers measure hematocrit by electrical conductivity and convert the hematocrit measurement to a total hemoglobin concentration, and some analyzers measure total hemoglobin concentration by spectroscopy, and convert the total hemoglobin concentration to a hematocrit value. Spectroscopic calibration algorithms can be developed to measure both hematocrit and total hemoglobin concentration.

Another analyte that resides inside red blood cells is folic acid (˜50% localized in red blood cells, the rest is stored mostly in the liver), and the measurement of RBC folate provides useful diagnostic information. Potassium is another analyte that resides in the RBCs, at about 20 times the concentration in plasma. However, measurement of RBC potassium provides no diagnostic value, whereas plasma potassium is a commonly ordered analyte for aiding in assessing acid-base-electrolyte balance.

Applying an unmetered sample volume to test strips is well known; some test strips contain absorbing sections that can accommodate a known volume of plasma, after the RBCs are retained in another section of the test strip near the blood application site. In some cases, the hematocrit affects the plasma flow in test strips, and therefore correction for hematocrit may improve accuracy of the analyte measurement. A common analyte that is measured using a test strip is blood glucose, and the test strips play a major role in managing diabetes.

POCT has improved patient care in several areas including the Emergency Department (ED) and Intensive Care Units (ICU) of hospitals, but the ED and ICU are usually very busy and may have space limitations for implementing more than one POCT analyzer. In addition to having accurate and reliable POCT in the ED, ICU, and for use by first responders, user friendliness is a major issue.

POCT analyzers are usually pre-calibrated, with calibration information installed in a barcoded label on the test strip or test cartridge. Examples of prior art are provided below in order to discuss some calibration issues. Spectroscopic calibration, for example calibration used for CO-oximetry, are more complex and are not discussed here. One or more calibrators (or calibration standards with known amounts of one or more analytes) may be used to calibrate a system. In the simplest cases of calibration, one or two calibrators are required. Commonly used calibration equations define a straight line, with signal response on the X-axis and concentration of analyte on the Y-axis. A straight line is usually defined by a slope and a Y-intercept (also referred to as an offset). Calibration adjustment for slope may be performed using two calibrators, and calibration adjustment for offset may be performed using one calibrator, referring to two-point and one-point calibration, respectively.

U.S. Pat. No. 5,096,669 to Lauks discloses a POCT cartridge for measuring blood gases and electrolytes in whole blood. The cartridge includes a preassembled calibration liquid (also referred to as calibration fluid) blister and a spike for rupturing the blister to release the calibration fluid, which is used to perform a one-point calibration of some of the electrochemical sensors in each cartridge. A screw and wedge mechanism are used to push the blister against the spike and force the released fluid into the electrochemical sensor chamber. The cartridge also comprises a hinged cap for covering the sample inlet after depositing sample in a sample well, and the cartridge does not include an optical chamber.

U.S. Pat. No. 7,094,330 to Lauks discloses another POCT cartridge for measuring blood gases and electrolytes in whole blood. This cartridge also includes a calibration fluid blister for performing a one-point calibration of some of the electrochemical sensors in each cartridge. The method of releasing the calibration fluid includes a plug for delaminating a section of the calibration fluid blister (a breakable seal 230). Also disclosed is a fill port 221 and a vent 222 for filling the calibration fluid blister. After filling the calibration fluid, a seal element 202 is laminated to seal off ports 221 and 222. A planar element comprising a plug 282 (for delaminating breakable seal 230) and a pin element 281 compresses the calibration fluid chamber 220 to release the calibration fluid. Blood must be loaded from a syringe, and the blood ejected from the syringe displaces the calibration fluid from the sensors. The syringe remains screwed to the cartridge inlet during measurement, therefore there is no requirement for a cap, and the cartridge does not include an optical chamber.

Pat. No. CA 2,978,737 to Samsoondar discloses another POCT cartridge for measuring blood gases, and electrolytes. Also disclosed in Pat. No. CA 2,978,737 is an optical chamber for performing spectroscopic measurement, for measuring CO-oximetry and bilirubin. Details of an example of the cartridges disclosed in Pat. No. CA 2,978,737 is provided in FIGS. 1A-1D of the present application. Capillary action is required to draw the blood sample through the optical chamber, up to an enlarged chamber outside the optical chamber. Calibration liquid from a blister is provided to perform a one-point calibration of some of the electrochemical sensors. Pressure on the dome portion of the blister pushes the blister against a spike, causing the bottom of the blister to rupture and release calibration fluid (may also be referred to as calibration liquid), and further pressure pushes released calibration liquid into the electrochemical sensor chamber. After a one-point calibration is performed, pressurized air from an air bladder pushes the blood into the electrochemical sensor chamber, displacing the calibration liquid. A screw cap is required to close the sample inlet. FIG. 1A illustrates how calibration liquid is able to flow to the top of the second housing member. A screw cap disclosed in Pat. No. CA 2,978,737 is not user friendly, and more user-friendly capping systems are needed. There is also a need to reduce the cost of POCT single-use cartridges, and at the same time, increase the test menu.

A major limitation of POCT blood gas and electrolyte systems disclosed in U.S. Pat. Nos. 5,096,669 and 7,094,330 is that their measurement technique is based on electrochemical sensors and therefore cannot measure CO-oximetry or Bilirubin, which can only be measured by spectroscopy. Oxygen is carried in the blood in two forms: (1) Dissolved in plasma and RBC water, which accounts for only 1-2% of the total blood oxygen content; and (2) Reversibly bound to hemoglobin, which accounts for about 98% of the total blood oxygen content. Partial pressure of oxygen (pO₂) is proportional to the quantity of oxygen dissolved in blood and is related to SO₂ (hemoglobin saturated with oxygen) through a sigmoidal curve (SO₂ plotted on the Y-axis and pO₂ plotted on the X-axis) referred to as the Oxygen-Hemoglobin Dissociation Curve. Measurement cartridges disclosed in U.S. Pat. Nos. 5,096,669, and 7,094,330 estimate SO₂ from measured pO₂, and estimate Hemoglobin (Hb) from measured Hematocrit. The Hb could be underestimated, possibly leading to unnecessary blood transfusion. CO-oximetry is the gold standard for measuring SO₂ because it actually measures % Oxy-Hb and % Deoxy-Hb, as well as % non-functional Hb like Met-Hb and Carboxy-Hb. A finger clip-on device referred to as a Pulse Oximeter is used in the ICU to measure SO₂ by a technique referred to as Pulse Oximetry, which may be inaccurate in the presence of elevated non-functional Hb. Measurement of Carboxy-Hb is essential for detecting carbon monoxide poisoning and monitoring treatment. Carbon monoxide poisoning could occur during excessive smoke inhalation. Measurement of Met-Hb is essential for detecting and treating elevated levels of Met-Hb, which could occur after ingestion of certain chemicals, in patients with certain enzyme deficiency, and in babies treated with nitric oxide for respiratory distress.

The inclusion of a calibration liquid blister within the test cartridges disclosed in U.S. Pat. Nos. 5,096,669, 7,094,330 and CA Pat. No. 2,978,737 adds significant cost to the cartridges, precluding their use in underdeveloped countries, and the calibration liquid in the blister can only perform a one-point calibration, and assumes that the slope of the calibration equation did not change. There is a need for simpler and less expensive POCT blood gas and electrolyte cartridges, and a system capable of performing more than just a one-point calibration. There is also a need for POCT cartridges that can also provide CO-oximetry and bilirubin without adding any significant cost to the cartridges. Bilirubin is a waste product of hemoglobin degradation, and elevated levels cause a condition known as jaundice. More than half of healthy neonates develop neonatal jaundice within days of birth because the baby's liver has not developed sufficiently to eliminate bilirubin from the blood. Babies with neonatal jaundice can easily be treated successfully, but if left untreated, neonatal jaundice could cause permanent brain damage and deafness.

Laboratory blood gas analyzers have evolved over the years. Since the eighties, companies began to add CO-oximetry, and later Bilirubin, to their blood gas menu. Because of the clinical need for CO-oximetry, laboratory blood gas analyzers without CO-oximetry are now virtually obsolete, and there is a need for POCT blood gas analyzers with single-use measurement cartridges to evolve like laboratory blood gas analyzers.

### SUMMARY OF THE INVENTION

The invention relates to a point-of-care testing (POCT) system. In various aspects, the invention relates to a system for measuring one or more properties of a blood sample, the system comprising a measurement cartridge for measuring the one or more properties of the blood sample, the measurement cartridge comprising: a measurement cartridge body having an upper surface and a lower surface, the upper surface defining a sample storage well for receiving the blood sample; a measurement electrochemical sensor chamber located within the measurement cartridge body, the measurement electrochemical sensor chamber comprising at least one first electrochemical sensor for generating measurement electrical signals in response to the one or more properties of the blood sample; and a blood flow conduit for establishing fluid communication between the sample storage well and the measurement electrochemical sensor chamber; a calibration cartridge comprising: a calibration cartridge body having an upper surface and a lower surface; at least one sealed blister within the calibration cartridge body containing calibration liquid comprising known amounts of the one or more properties; and a calibration electrochemical sensor chamber located within the calibration cartridge body, the calibration electrochemical sensor chamber comprising at least one second electrochemical sensor for generating calibration electrical signals in response to the calibration liquid, wherein the at least one first and second electrochemical sensors generate similar electrical signals in response to the same amount of the same one or more properties; and a calibration liquid conduit for establishing fluid communication between the at least one sealed blister and the calibration electrochemical sensor chamber; and an analyzer comprising: a receptor for separately receiving the calibration cartridge and the measurement cartridge; means for releasing calibration liquid from the at least one sealed blister containing the calibration liquid; means for moving the calibration liquid from the at least one sealed blister to the at least one second electrochemical sensor of the calibration cartridge; means for moving the blood from the sample storage well to the at least one first electrochemical sensor of the measurement cartridge; an electrical receiver for receiving the calibration electrical signals generated by the at least one second electrochemical sensor and for receiving the measurement electrical signals generated by the least one first electrochemical sensor; and a processor for developing a mathematical relation between the calibration electrical signals and the one or more properties in the calibration liquid, and applying the mathematical relation to the measurement electrical signals to determine the amount of the one or more properties in the blood sample.

In various embodiments, the at least one sealed blister consists of one sealed blister containing calibration liquid, for performing one-point calibration of the at least one first electrochemical sensor.

In various embodiments, the at least one sealed blister consists of two sealed blisters containing calibration liquid, for performing two-point calibration of the at least one first electrochemical sensor.

In various embodiments, the system comprises a plurality of calibration cartridges for performing multi-point calibration, and each of the plurality of calibration cartridges comprises a single calibration liquid blister in order to provide a plurality of calibration liquid blisters, and wherein each of the plurality of calibration liquid blisters comprises a different liquid composition.

In various embodiments, the one or more properties of the blood sample is pH and the at least one first electrochemical sensor and the at least one second electrochemical sensor are potentiometric electrochemical sensors.

In various embodiments, the measurement cartridge further comprises an optical chamber having at least one of an upper optical window and a lower optical window, the optical chamber in fluid communication with the blood flow conduit, the optical chamber for facilitating interrogation of a portion of the blood sample by electromagnetic radiation, for measuring one or more other properties of the blood.

In various embodiments, the means for moving the blood sample from the sample storage well to the at least one first electrochemical sensor of the measurement cartridge comprises at least one of: an air bladder disposed in the measurement cartridge body, the air bladder in fluid communication with the sample storage well; an analyzer pump attachable to a duct of the measurement cartridge body and in fluid communication with the sample storage well; a surface of the blood flow conduit sufficiently hydrophilic to promote blood flow by capillary action; a cap for covering the sample storage well; and at least one vent defined by a surface in the cartridge body or the cap in communication with the blood flow conduit.

In various embodiments, the measurement cartridge further comprises one or more reagents and means for mixing the blood sample and the one or more reagents.

In various embodiments, the sample storage well comprises a top portion for receiving the blood sample and a bottom portion for releasing at least a portion of the blood sample to the blood flow conduit, and wherein the measurement cartridge further comprises means for mitigating blood flow out of the bottom portion of the sample storage well when blood is received in the sample storage well through the top portion.

In various embodiments, the measurement cartridge further comprises a cap, the cap selected from a hinged cap, a pivotal cap, a sliding cap, and a screw cap for covering the sample storage well.

In various embodiments, the at least one first electrochemical sensor and the at least one second electrochemical sensor are of the same type manufactured in the same batch.

In another aspect, the invention relates to a calibration cartridge for calibrating at least one electrochemical sensor used for measuring one or more properties of a blood sample, the calibration cartridge comprising: a calibration cartridge body having an upper surface and a lower surface; at least one sealed blister located within the calibration body and containing a calibration liquid, wherein the calibration liquid comprises a known amount of the one or more properties of the blood sample; means for releasing the calibration liquid from the at least one sealed blister; a first calibration liquid conduit in fluid communication with each of the at least one sealed blister for receiving the calibration liquid; a second calibration liquid conduit for receiving calibration liquid from each first calibration liquid conduit, wherein the second calibration conduit is closed off from any other liquid influx; an electrochemical sensor chamber in fluid communication with the second calibration liquid conduit, the electrochemical sensor chamber comprising at least one electrochemical sensor and at least one electrical output, when installed with an associated analyzer, the at least one electrical output is configured to make contact with at least one electrical input of the associated analyzer, used to measure the one or more properties of the blood sample; and a vent in communication with the electrochemical sensor chamber, wherein the vent is for releasing pressure and allowing the calibration liquid to make contact with the at least one electrochemical sensor.

In various embodiments, the calibration cartridge body does not include a sample storage well.

In various embodiments, the calibration cartridge comprises one sealed blister containing calibration liquid, for performing one-point calibration of the at least one electrochemical sensor.

In various embodiments, the calibration cartridge comprises two sealed blisters containing different calibration liquids, two first calibration liquid conduits, and one second calibration liquid conduit, for performing two-point calibration of the at least one electrochemical sensor. In various embodiments, the calibration cartridge may comprise a directional valve disposed at the junction of the two first calibration liquid conduits and the second calibration liquid conduit.

In various embodiments, the means for releasing calibration liquid comprise: (a) at least one spike for rupturing the at least one sealed blister; or (b) a weakened portion of each of the at least one sealed blister for rupturing the at least one sealed blister, wherein when the calibration cartridge is installed with an associated analyzer, a force on the at least one sealed blister is provided by the associated analyzer.

In another aspect, the invention relates to a measurement cartridge for measuring one or more properties of a blood sample, the measurement cartridge comprising: a cartridge body comprising an upper surface and a lower surface, the upper surface defining a sample storage well having a top portion for receiving the blood sample, and a bottom portion for releasing at least a portion of the blood sample into one or more blood conduits; one or more detection chambers for receiving blood from the one or more blood conduits and providing signals for measuring the one or more properties of the blood; a cap hingeably attached to the cartridge body and adjustable from a first position to a second position, the cap comprising a top side and an underside, the underside comprising a plunger configured to enter the sample storage well; in the cap first position the measurement cartridge is configured to receive the blood sample in the sample storage well; in the cap second position the cartridge is configured with the plunger inserted in the sample storage well, the plunger displacing at least some of the blood sample into the one or more blood conduits; and at least one vent for releasing pressure in the one or more detection chambers.

A detection chamber is a chamber containing at least some of the blood sample, wherein the analyte in the blood sample, when in the detection chamber, provides a measurable signal. In various embodiments, the signal may be: a) an electrical signal from an electrochemical sensor disposed in the detection chamber, when the blood sample makes contact with the electrochemical sensor, or b) electromagnetic radiation (EMR) emerging from the blood sample in the detection chamber, after EMR from a source in an associated analyzer impinges upon the blood sample in the detection chamber. The EMR not absorbed or scattered by the blood sample is detected by a photodetector in the associated analyzer.

In various embodiments, the one or more detection chambers comprise an electrochemical sensor chamber having at least one electrochemical sensor.

In various embodiments, the at least one electrochemical sensor is one of an amperometric sensor, a conductivity sensor and a potentiometric sensor.

In various embodiments, the one or more properties of blood is pH, and the electrochemical sensor is a potentiometric sensor.

In various embodiments, the measurement cartridge further comprises one or more reagents in communication with the one or more blood conduits and means for mixing the blood sample and one or more reagents to produce altered blood.

In various embodiments, the one or more detection chambers comprise an optical chamber having at least one of an upper optical window and a lower optical window, the optical chamber for facilitating interrogation of the blood sample or the altered blood by electromagnetic radiation.

In various embodiments, the one or more detection chambers comprise an electrochemical sensor chamber having at least one electrochemical sensor and an optical chamber having at least one of an upper optical window and a lower optical window, the optical chamber for facilitating interrogation of the blood sample by electromagnetic radiation. The measurement cartridge may further comprise one or more reagents in communication with the one or more blood conduits for mixing the blood sample and one or more reagents to produce altered blood.

In a further aspect, the invention relates to a system for measuring one or more properties of a blood sample, the system comprising a measurement cartridge as described herein and an analyzer, the analyzer comprising: a receptor for receiving the measurement cartridge; at least one source of interrogating electromagnetic radiation (EMR) for interrogating at least some of the blood sample when the blood sample is positioned within the optical chamber, or for interrogating at least some of the altered blood when the altered blood sample is positioned within the optical chamber; at least one of: a one-dimensional multi-channel detector for receiving EMR emerging from one of the blood sample in the optical chamber or the altered blood sample in the optical chamber, via an EMR dispersing element, the EMR dispersing element for providing wavelength-specific EMR and the one-dimensional multi-channel detector for generating wavelength-specific electrical signals, or a two-dimensional multi-channel detector for receiving EMR emerging from one of the blood sample in the optical chamber or the altered blood sample in the optical chamber, and generating detector-specific electrical signals; one or more analog to digital converters for receiving one or more of the wavelength-specific electrical signals for generating wavelength-specific digital information, or the detector-specific electrical signals for generating detector-specific digital information; and one or more processors for controlling the analyzer and transforming at least one of the wavelength-specific digital information and the detector-specific digital information into the one or more properties of the blood sample.

Other aspects and features of the present invention will become apparent to those having ordinary skill in the art, upon review of the following description of specific embodiments of the invention, which are provided as non-limiting examples.

### BRIEF DESCRIPTION OF THE DRAWINGS

A better understanding of the novel features and advantages of the present invention will be made by reading the detailed description of the preferred embodiments provided later, in conjunction with the accompanying drawings, in which:

FIG. 1A (Prior Art) is an exploded view illustrating a version of a cartridge comprising an optical chamber, electrochemical sensors, and a blister containing calibration liquid for calibrating at least one of the electrochemical sensors;

FIG. 1B (Prior Art) is a perspective top view of the cartridge illustrated in FIG. 1A, with sample inlet that works in conjunction with a screw cap;

FIG. 1C (Prior Art) is a perspective bottom view of the cartridge illustrated in FIG. 1A;

FIG. 1D (Prior Art) is a detailed view of detail D shown in FIG. 1A, illustrating that the calibration liquid conduit is not closed (i.e., it is open to an influx of blood);

FIG. 2A is an exploded perspective top view of a measurement cartridge 10a for measuring at least one property of blood, according to a first embodiment of a measurement cartridge;

FIG. 2B is a bottom view of the first housing member 30a of the measurement cartridge shown in FIG. 2A;

FIG. 2C is the bottom view of the first housing member 30a of the measurement cartridge shown in FIG. 2B, overlaid by and in alignment with a gasket 100a shown in FIG. 2A;

FIG. 2D is a top view of the second housing member 40a of the measurement cartridge shown in FIG. 2A;

FIG. 2E is the top view of the second housing member 40a shown in FIG. 2D, overlaid by and in alignment with the gasket 100a shown in FIG. 2A;

FIG. 2F is a perspective top view of the measurement cartridge 10a shown in FIG. 2A, in an open configuration;

FIG. 2G is a perspective bottom view of the measurement cartridge 10a shown in FIG. 2F;

FIG. 3A is top view of the measurement cartridge 10a shown in FIG. 2A, in an open configuration;

FIG. 3B is top view of the cartridge 10a shown in FIG. 2A, in a closed configuration;

FIG. 3C is an enlarged cross-sectional view through the cartridge 10a shown in FIG. 3A along line C-C;

FIG. 3D is an enlarged cross-sectional view through the cartridge 10a shown in FIG. 3B along line D-D;

FIG. 3E is an enlarged cross-sectional view through the cartridge 10a shown in FIG. 3B along line E-E;

FIG. 3F is a detailed view of detail F of the bottom portion of the sample storage well shown in FIG. 3E;

FIG. 4A is an exploded perspective top view of a calibration cartridge 20a for calibrating one or more electrochemical sensors, according to a first embodiment of a calibration cartridge;

FIG. 4B is a bottom view of the first housing member 50a of the calibration cartridge shown in FIG. 4A;

FIG. 4C is the bottom view of the first housing member 50a of the calibration cartridge shown in FIG. 4B, overlaid by and in alignment with a gasket 102a shown in FIG. 4A;

FIG. 4D is a top view of the second housing member 60a of the calibration cartridge shown in FIG. 4A;

FIG. 4E is the top view of the second housing member 60a shown in FIG. 4D, overlaid by and in alignment with the gasket 102a shown in FIG. 4A;

FIG. 4F is a perspective top view of the calibration cartridge 20a shown in FIG. 4A;

FIG. 4G is a perspective bottom view of the calibration cartridge 20a shown in FIG. 4A, with the bottom laminate 99a removed;

FIG. 5A is a top view of the calibration cartridge 20a shown in FIG. 4A;

FIG. 5B is an enlarged cross-sectional view through the calibration cartridge 20a shown in FIG. 5A along line B-B;

FIG. 5C is an enlarged cross-sectional view through the calibration cartridge 20a shown in FIG. 5A along line C-C;

FIG. 5D is an enlarged cross-sectional view through the calibration cartridge 20a shown in FIG. 5A along line D-D;

FIG. 6A is an exploded perspective top view of a calibration cartridge 20b for calibrating one or more electrochemical sensors, according to a second embodiment of a calibration cartridge;

FIG. 6B is a perspective top view of the calibration cartridge 20b shown in FIG. 6A;

FIG. 6C is a perspective bottom view of the calibration cartridge 20b shown in FIG. 6A, with the bottom laminate 99b removed;

FIG. 7A is a top view of the calibration cartridge 20b shown in FIG. 6A;

FIG. 7B is a detailed view of detail B of the calibration cartridge 20b shown in FIG. 7A;

FIG. 7C is a perspective view of a directional valve element 69b of calibration cartridge 20b, which for example, could be an elastomeric flap;

FIG. 7D is an enlarged cross-sectional view through the calibration cartridge 20b shown in FIG. 7A along line D-D;

FIG. 7E is an enlarged cross-sectional view through the calibration cartridge 20b shown in FIG. 7A along line E-E;

FIG. 8A is a perspective top view of the second housing member 60b of the calibration cartridge 20b shown in FIG. 6A;

FIG. 8B is a perspective top view of the second housing member 60b of the calibration cartridge 20b shown in FIG. 8A, with directional valve element 69b inserted in a nest 64b shown in FIG. 8E;

FIG. 8C is a perspective bottom view of the first housing member 50b of the calibration cartridge 20b shown in FIG. 6A;

FIG. 8D is a perspective bottom view of the first housing member 50b of the calibration cartridge 20b shown in FIG. 8C, overlaid with and in alignment with gasket 102b, and in alignment with directional valve element 69b (which is usually inserted in the nest 64b);

FIG. 8E is a detailed view of detail E of second housing member 60b of calibration cartridge 20b shown in FIG. 8A;

FIG. 8F is a detailed view of detail F of second housing member 60b of calibration cartridge 20b shown in FIG. 8B;

FIG. 8G is a detailed view of detail G of first housing member 50b of calibration cartridge 20b shown in FIG. 8C;

FIG. 8H is a detailed view of detail H of first housing member 50b of calibration cartridge 20b shown in FIG. 8D;

FIG. 9A is an exploded perspective top view of a measurement cartridge 10b for measuring at least one property of blood, according to a second embodiment of a measurement cartridge;

FIG. 9B is a bottom view of the first housing member 30b of the measurement cartridge shown in FIG. 9A;

FIG. 9C is the bottom view of the first housing member 30b of the measurement cartridge shown in FIG. 9B, overlaid by and in alignment with a gasket 100b shown in FIG. 9A;

FIG. 9D is a top view of the second housing member 40b of the measurement cartridge shown in FIG. 9A;

FIG. 9E is the top view of the second housing member 40b shown in FIG. 9D, overlaid by and in alignment with the gasket 100b shown in FIG. 9A;

FIG. 9F is a perspective top view of the measurement cartridge 10b shown in FIG. 9A, in an open configuration;

FIG. 9G is a perspective bottom view of the measurement cartridge 10b shown in FIG. 9F;

FIG. 10A is an exploded perspective top view of a measurement cartridge 10c for measuring at least one property of blood, according to a third embodiment of a measurement cartridge;

FIG. 10B is a perspective top view of the measurement cartridge 10c shown in FIG. 10A, in an open configuration;

FIG. 10C is a perspective bottom view of the measurement cartridge 10c shown in FIG. 10B;

FIG. 10D is a top view of the measurement cartridge 10c shown in FIG. 10A, in a closed configuration;

FIG. 10E is an enlarged cross-sectional view through the measurement cartridge 10c shown in FIG. 10D along line E-E;

FIG. 10F is an enlarged cross-sectional view through the measurement cartridge 10c shown in FIG. 10D along line F-F;

FIG. 10G is an enlarged cross-sectional view through the measurement cartridge 10c shown in FIG. 10D along line G-G;

FIG. 11A is an exploded perspective top view of a measurement cartridge 10d for measuring at least one property of blood, according to a fourth embodiment of a measurement cartridge;

FIG. 11B is a bottom view of the first housing member 30d of the measurement cartridge shown in FIG. 11A;

FIG. 11C is the bottom view of the first housing member 30d of the measurement cartridge shown in FIG. 11B, overlaid by and in alignment with a gasket 100d shown in FIG. 11A;

FIG. 11D is a top view of the second housing member 40d of the measurement cartridge shown in FIG. 11A;

FIG. 11E is the top view of the second housing member 40d shown in FIG. 11D, overlaid by and in alignment with the gasket 100d shown in FIG. 11A;

FIG. 11F is a top view of the measurement cartridge 10d shown in FIG. 11A, in an open configuration;

FIG. 11G is an enlarged cross-sectional view through the measurement cartridge 10d shown in FIG. 11F along line G-G;

FIG. 12A is a top view of the measurement cartridge 10d shown in FIG. 11A, in a closed configuration;

FIG. 12B is a bottom view of the measurement cartridge 10d shown in FIG. 11A;

FIG. 12C is an enlarged cross-sectional view through the measurement cartridge 10d shown in FIG. 12A along line C-C;

FIG. 12D is an enlarged cross-sectional view through the measurement cartridge 10d shown in FIG. 12A along line D-D;

FIG. 13A is an exploded perspective top view of a measurement cartridge 10e for measuring at least one property of blood, according to a fifth embodiment of a measurement cartridge;

FIG. 13B is a bottom view of the first housing member 30e of the measurement cartridge shown in FIG. 13A;

FIG. 13C is the bottom view of the first housing member 30e of the measurement cartridge shown in FIG. 13B, overlaid by and in alignment with a gasket 100e shown in FIG. 13A;

FIG. 13D is a top view of the second housing member 40e of the measurement cartridge shown in FIG. 13A;

FIG. 13E is the top view of the second housing member 40a shown in FIG. 13D, overlaid by and in alignment with the gasket 100e shown in FIG. 13A;

FIG. 13F is a perspective top view of the cartridge 10e shown in FIG. 13A, in a closed configuration;

FIG. 13G is a perspective bottom view of the measurement cartridge 10e shown in FIG. 13A;

FIG. 14A is top view of the measurement cartridge 10e shown in FIG. 13A, in an open configuration;

FIG. 14B is an enlarged cross-sectional view through the measurement cartridge 10e shown in FIG. 14A along line B-B;

FIG. 14C is top view of the measurement cartridge 10e shown in FIG. 13A, in a closed configuration;

FIG. 14D is an enlarged cross-sectional view through the measurement cartridge 10e shown in FIG. 14C along line D-D;

FIG. 14E is a detailed view of detail E of measurement cartridge 10e shown in FIG. 14D;

FIG. 14F is a detailed view of detail F of measurement cartridge 10e shown in FIG. 14A;

FIG. 15 is a block diagram of an example of a system 70 (lower panel) for measuring one or more analyte quantities per unit volume of blood and one or more formed element quantities per unit volume of blood, in a blood sample, and output displays of the system (upper left and right panels) are provided as non-limiting examples;

FIG. 16A is an exploded perspective top view of a measurement cartridge 10f for measuring at least one property of blood, according to a sixth embodiment of a measurement cartridge;

FIG. 16B is a bottom view of the first housing member 30f of the measurement cartridge shown in FIG. 16A;

FIG. 16C is the bottom view of the first housing member 30f of the measurement cartridge shown in FIG. 16B, overlaid by and in alignment with a gasket 100f shown in FIG. 16A;

FIG. 16D is a top view of the second housing member 40f of the measurement cartridge shown in FIG. 16A;

FIG. 16E is the top view of the second housing member 40f shown in FIG. 16D, overlaid by and in alignment with the gasket 100f shown in FIG. 16A;

FIG. 16F is a perspective top view of the measurement cartridge 10f shown in FIG. 16A, in an open configuration;

FIG. 16G is a perspective bottom view of the measurement cartridge 10f shown in FIG. 16A;

FIG. 17A is a top view of the measurement cartridge 10f shown in FIG. 16A, in a closed configuration;

FIG. 17B is an enlarged cross-sectional view through the measurement cartridge 10f shown in FIG. 17A along line B-B;

FIG. 17C is a detailed view of detail C of measurement cartridge 10f shown in FIG. 17B;

FIG. 17D is a detailed view of detail D of measurement cartridge 10f shown in FIG. 17C;

FIG. 18A is a perspective top view of a calibration cartridge 20b and an associated analyzer 80, having a receptor 14 for receiving measurement cartridge 20b;

FIG. 18B is a perspective top view of a measurement cartridge 10b and the associate analyzer 80 shown in FIG. 18A;

FIG. 18C is a perspective top view of the measurement cartridge 10b inserted in the slot 14 of the associated analyzer 80, shown in FIG. 18B;

FIG. 19A is an exploded perspective top view of a measurement cartridge 10g for measuring at least one property of blood, according to a seventh embodiment of a measurement cartridge;

FIG. 19B is a bottom view of the first housing member 30g of the measurement cartridge shown in FIG. 19A;

FIG. 19C is the bottom view of the first housing member 30g of the measurement cartridge shown in FIG. 19B, overlaid by and in alignment with the gasket 100g shown in FIG. 19A;

FIG. 19D is a top view of the second housing member 40g of the measurement cartridge shown in FIG. 19A;

FIG. 19E is the top view of the second housing member 40g shown in FIG. 19D, overlaid by and in alignment with the gasket 100g shown in FIG. 19A;

FIG. 19F is a perspective top view of the measurement cartridge 10g shown in FIG. 19A, in an open configuration;

FIG. 19G is a perspective bottom view of the measurement cartridge 10g shown in FIG. 19A;

FIG. 20A is a top view of the measurement cartridge 10g shown in FIG. 19A, in a closed configuration;

FIG. 20B is a perspective top view of directional valve element 67g;

FIG. 20C is a perspective top view of directional valve element 68g;

FIG. 20D is an enlarged cross-sectional view through the measurement cartridge 10g shown in FIG. 20A along line D-D;

FIG. 20E is an enlarged cross-sectional view through the measurement cartridge 10g shown in FIG. 20A along line E-E;

FIG. 20F is an enlarged cross-sectional view through the measurement cartridge 10g shown in FIG. 20A along line F-F;

FIG. 20G is an enlarged cross-sectional view through the measurement cartridge 10g shown in FIG. 20A along line G-G;

FIG. 21A is a perspective top view of the second housing member 40g of the measurement cartridge 10g shown in FIG. 19A;

FIG. 21B is the perspective top view of the second housing member 40g of the measurement cartridge 10g shown in FIG. 21A, showing directional valve elements 67g and 68g seated in their respective nests 65g and 66g;

FIG. 21C is the perspective top view of the second housing member 40g of the measurement cartridge 10g shown in FIG. 21B, overlaid by and in alignment with the gasket 100g shown in FIG. 19A;

FIG. 21D is a perspective bottom view of the first housing member 30g of the measurement cartridge 10g shown in FIG. 19A;

FIG. 21E is a detailed view of detail E of second housing member 40g of measurement cartridge 10g shown in FIG. 21A;

FIG. 21F is a detailed view of detail F of second housing member 40g of measurement cartridge 10g shown in FIG. 21B;

FIG. 21G is a detailed view of detail G of second housing member 40g of measurement cartridge 10g shown in FIG. 21C;

FIG. 21H is a detailed view of detail H of first housing member 30g of measurement cartridge 10g shown in FIG. 21D;

FIG. 21J is a detailed view of detail J of first housing member 30g of measurement cartridge 10g shown in FIG. 21D;

FIG. 22A is an exploded perspective top view of a measurement cartridge 10h for measuring at least one property of blood, according to an eighth embodiment of a measurement cartridge;

FIG. 22B is a bottom view of the first housing member 30h of the measurement cartridge shown in FIG. 22A;

FIG. 22C is the bottom view of the first housing member 30h of the measurement cartridge shown in FIG. 22B, overlaid by and in alignment with a gasket 100h shown in FIG. 22A;

FIG. 22D is a top view of the second housing member 40h of the measurement cartridge shown in FIG. 22A;

FIG. 22E is the top view of the second housing member 40h shown in FIG. 22D, overlaid by and in alignment with the gasket 100h shown in FIG. 22A;

FIG. 22F is a perspective top view of the measurement cartridge 10h shown in FIG. 22A in an open configuration;

FIG. 22G is a perspective bottom view of the measurement cartridge 10h shown in FIG. 22A;

FIG. 23A is a top view of the measurement cartridge 10h shown in FIG. 22A, with the cap in a closed configuration;

FIG. 23B is an enlarged cross-sectional view through the measurement cartridge 10h shown in FIG. 23A along line B-B;

FIG. 23C is an enlarged cross-sectional view through the measurement cartridge 10h shown in FIG. 23A along line C-C; and

FIG. 23D is a detailed view of detail D of measurement cartridge 10h shown in FIG. 23C.

For a better understanding of the present invention, and to show more clearly how it may be carried into effect, reference will now be made, by way of example, to the accompanying drawings, and which are described in the following detailed description of preferred aspects of the invention.

### DETAILED DESCRIPTION OF PREFERRED ASPECTS OF THE INVENTION

POCT systems comprising an analyzer, a measurement cartridge having one or more electrochemical sensors in a detection chamber, and a calibration cartridge having one or more similar electrochemical sensors are described. Systems comprising measurement cartridges having no calibration liquid blisters, and calibration cartridges having one or two calibration liquid blisters for performing one-point calibration (for offset correction) or two-point calibration (offset and slope correction), respectively, are described. Also described are systems comprising measurement cartridges having one calibration liquid blister for performing one-point calibration and calibration cartridges having two calibration liquid blisters for performing two-point calibration. Although the examples of calibration cartridges illustrate one and two calibration liquid blisters for simplicity, any number of calibration liquid blisters are considered to be within the scope of the present application. Also described are measurement cartridges comprising one or more detection chambers, wherein the one or more detection chambers comprise one or more optical chambers.

In this application, two types of cartridges are described: 1) Calibration Cartridges, and 2) Measurement Cartridges. In the calibration cartridge, no sample storage well is required, wherein the calibration liquid conduit entering the electrochemical sensor conduit is closed off from any other liquid influx, like influx of blood. For illustration, two examples of calibration cartridges, 20a and 20b, are provided, and eight examples of measurement cartridges, 10a, 10b, 10c, 10d, 10e, 10f, 10g and 10h, are provided. Various combinations of detection chambers in the measurement cartridges are provided, in order to increase the versatility of the measurement cartridges.

As used herein, the terms “comprising,” “having,” “including” and “containing,” and grammatical variations thereof, are inclusive or open-ended and do not exclude additional, un-recited elements and/or method steps. The term “consisting essentially of” when used herein in connection with a use or method, denotes that additional elements and/or method steps may be present, but that these additions do not materially affect the manner in which the recited method or use functions. The term “consisting of” when used herein in connection with a use or method, excludes the presence of additional elements and/or method steps. A use or method described herein as comprising certain elements and/or steps may also, in certain embodiments consist essentially of those elements and/or steps, and in other embodiments consist of those elements and/or steps, whether or not these embodiments are specifically referred to. The term “plurality” as used herein means more than one, for example, two or more, three or more, four or more, and the like. Unless otherwise defined herein, all technical and scientific terms used herein have the same meaning as commonly understood by one of ordinary skill in the art. As used herein, the term “about” refers to an approximately +/−25% variation from a given value. It is to be understood that such a variation is always included in any given value provided herein, whether or not it is specifically referred to. The use of the word “a” or “an” when used herein in conjunction with the term “comprising” may mean “one,” but it is also consistent with the meaning of “one or more,” “at least one” and “one or more than one.”

The terms “operatively connected”, “in operative communication”, “in fluid communication”, “in fluid connection” or “fluidly connected” and the like, describe elements of the cartridges, for example, channels, ducts, conduits, tunnels, passageways, that permit either fluid flow, gas flow, or both fluid and gas flow between the various compartments or elements within the cartridge that are connected by the channels, ducts, conduits, tunnels, passageways and the like.

Detailed description of features of examples of the invention is described with reference to the accompanying drawings. These examples are to be considered non-limiting, and a person having ordinary skill in the art should understand that variations are within the scope of the invention, even though they are not explicitly illustrated. The same reference numerals are used for similar elements in different examples; in some cases, letters are appended to the end of the reference numerals to denote the embodiment of the invention illustrated. For example, 10a and 10b refer to two different examples of a Measurement Cartridge, and 20a and 20b refer to two different examples of a Calibration Cartridge. To maintain the distinction between a Measurement Cartridge and a Calibration Cartridge, attempts are made to provide different reference numerals for similar structures in the two different types of cartridges. It should be noted that absence of a letter after a reference numeral may refer to a structural feature of the invention incorporated in multiple examples. For easy reference, Table 1 provides a list of the reference numerals used, and a brief description of the corresponding structural features.

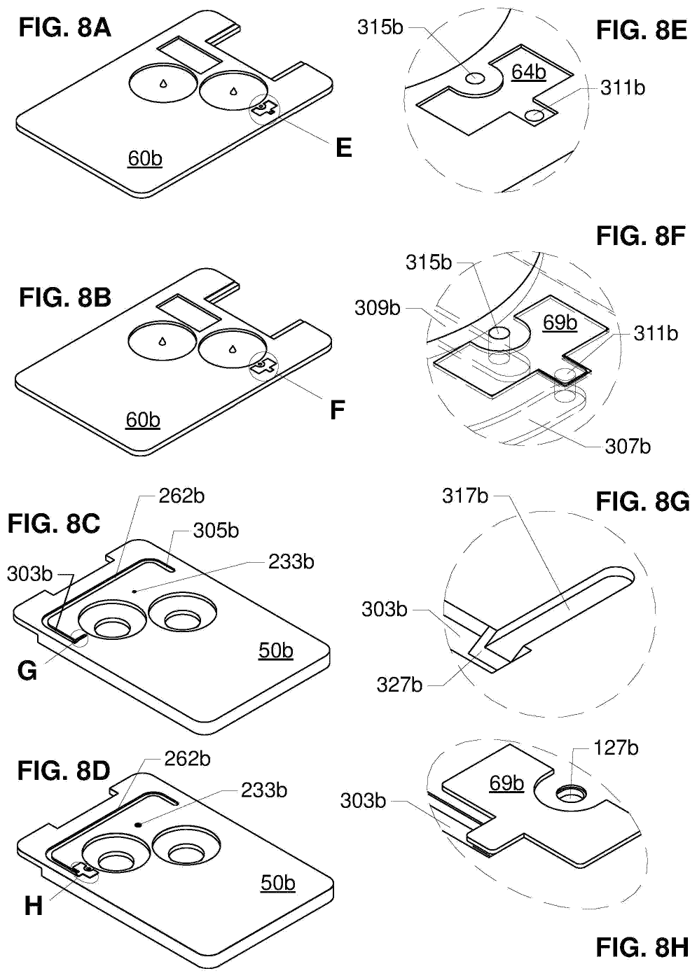

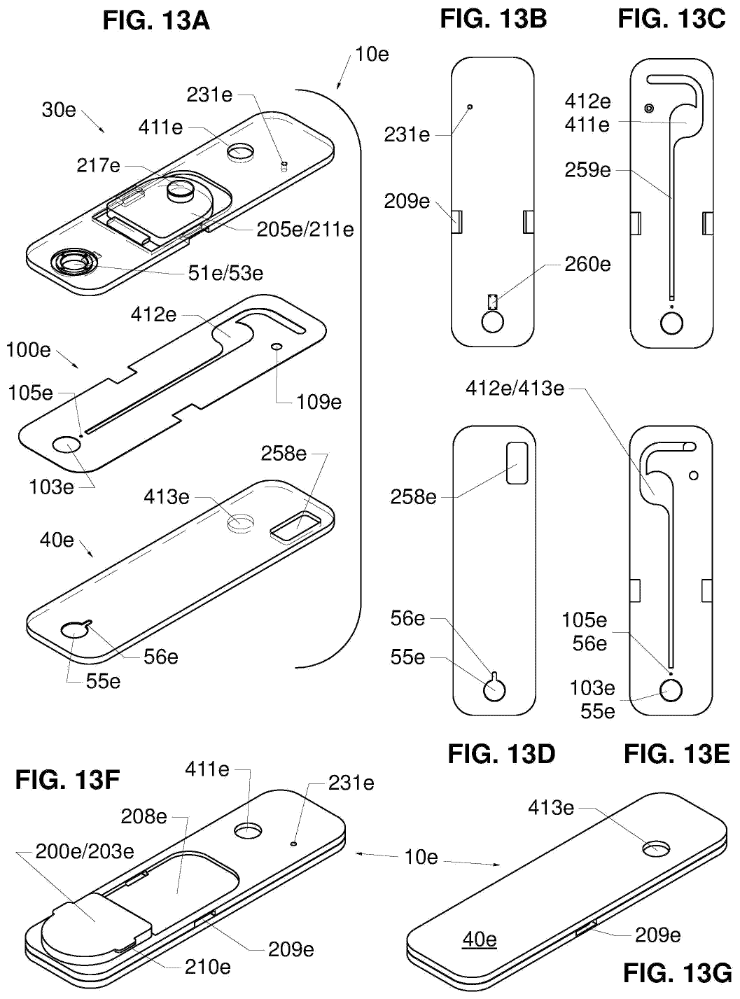

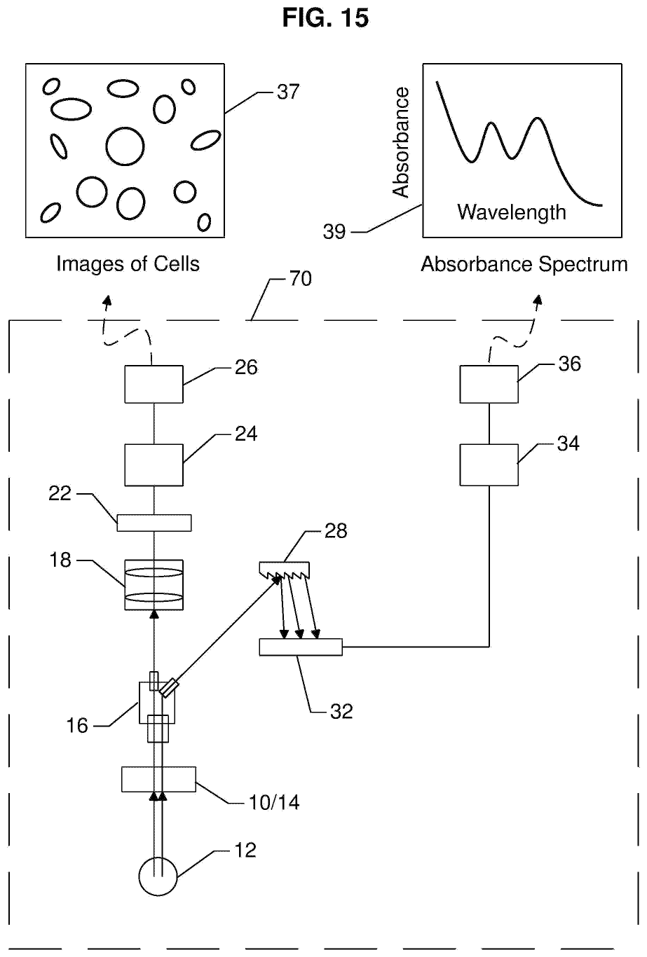

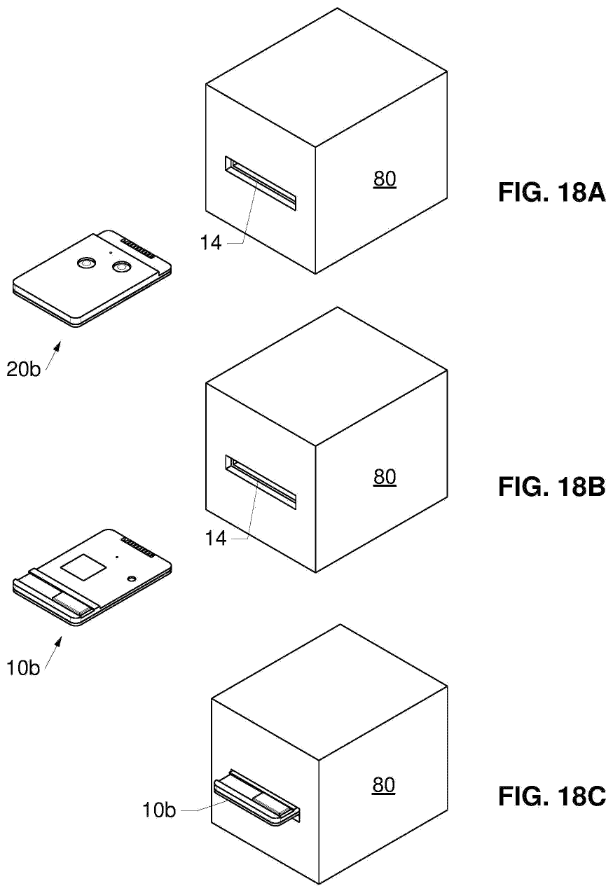

TABLE 1
Description of Structural Features.

| Reference | |
|---|---|
| Numerals | Description of Structural Features |
| 10 | Generic measurement cartridge having an optical chamber, depicted |
| | in FIG. 15 |
| 10a | First embodiment of a measurement cartridge |
| 10b | Second embodiment of a measurement cartridge |
| 10c | Third embodiment of a measurement cartridge |
| 10d | Fourth embodiment of a measurement cartridge |
| 10e | Fifth embodiment of a measurement cartridge |
| 10f | Sixth embodiment of a measurement cartridge |
| 10g | Seventh embodiment of a measurement cartridge |
| 10h | Eighth embodiment of a measurement cartridge |
| 12 | Source of electromagnetic radiation (EMR) of an analyzer of system |
| | 70 |
| 14 | Generic receptor in an analyzer of system 70 for receiving a cartridge |
| | depicted in FIG. 15, and analyzer 80 depicted in FIGS. 18A-18C |
| 16 | Beam splitter of an analyzer of system 70 (bifurcated optical fiber |
| | shown as an example) |
| 18 | Magnifying system of an analyzer of system 70 |
| 20a | First embodiment of a calibration cartridge |
| 20b | Second embodiment of a calibration cartridge |
| 22 | Two-dimensional multi-channel detector of an analyzer of system 70 |
| 24 | Analog to digital converter of an analyzer of system 70 |
| 26 | Processor of an analyzer of system 70 |
| 28 | EMR dispersing element, e.g. a grating or a prism (a grating shown) |
| 30a | First housing member of measurement cartridge 10a |
| 30b | First housing member of measurement cartridge 10b |
| 30c | First housing member of measurement cartridge 10c |
| 30d | First housing member of measurement cartridge 10d |
| 30e | First housing member of measurement cartridge 10e |
| 30f | First housing member of measurement cartridge 10f |
| 30g | First housing member of measurement cartridge 10g |
| 30h | First housing member of measurement cartridge 10h |
| 32 | One-dimensional multi-channel detector of analyzer 70 |
| 34 | Analog to digital converter of an analyzer of system 70 |
| 36 | Processor of an analyzer of system 70 |
| 37 | Example of a display of two-dimensional detector 22 |
| 39 | Example of a display of one-dimensional detector 32 |
| 40a | Second housing member of measurement cartridge 10a |
| 40b | Second housing member of measurement cartridge 10b |
| 40c | Second housing member of measurement cartridge 10c |
| 40d | Second housing member of measurement cartridge 10d |
| 40e | Second housing member of measurement cartridge 10e |
| 40f | Second housing member of measurement cartridge 10f |
| 40g | Second housing member of measurement cartridge 10g |
| 40h | Second housing member of measurement cartridge 10h |
| 50a | First housing member of calibration cartridge 20a |
| 50b | First housing member of calibration cartridge 20b |
| 51a | Sample storage well of measurement cartridge 10a |
| 51b | Sample storage well of measurement cartridge 10b |
| 51c | Sample storage well of measurement cartridge 10c |
| 51d | Sample storage well of measurement cartridge 10d |
| 51e | Sample storage well of measurement cartridge 10e |
| 51f | Sample storage well of measurement cartridge 10f |
| 51g | Sample storage well of measurement cartridge 10g |
| 51h | Sample storage well of measurement cartridge 10h |
| 53a | Top opening (or top portion) of a sample storage well 51a |
| 53b | Top opening (or top portion) of a sample storage well 51b |
| 53c | Top opening (or top portion) of a sample storage well 51c |
| 53e | Top opening (or top portion) of a sample storage well 51e |
| 53f | Top opening (or top portion) of a sample storage well 51f |
| 53g | Top opening (or top portion) of a sample storage well 51g |
| 53h | Top opening (or top portion) of a sample storage well 51h |
| 55a | Bottom opening (or bottom portion) of a sample storage well 51a |
| 55b | Bottom opening (or bottom portion) of a sample storage well 51b |
| 55c | Bottom opening (or bottom portion) of a sample storage well 51c |
| 55e | Bottom opening (or bottom portion) of a sample storage well 51e |
| 55f | Bottom opening (or bottom portion) of a sample storage well 51f |
| 55g | Bottom opening (or bottom portion) of a sample storage well 51g |
| 55h | Bottom opening (or bottom portion) of a sample storage well 51h |
| 56a | Extension of the bottom opening 55a of sample storage well 51a of |
| | cartridge 10a for connecting sample storage well 51a to blood flow |
| | conduit 259a |
| 56b | Extension of the bottom opening 55b of sample storage well 51b of |
| | cartridge 10b for connecting sample storage well 51b to blood flow |
| | conduit 259b |
| 56e | Extension of the bottom opening 55e of sample storage well 51e of |
| | cartridge 10e for connecting sample storage well 51e to blood flow |
| | conduit 259e |
| 56f | Extension of the bottom opening 55f of sample storage well 51f of |
| | cartridge 10f for connecting sample storage well 51f to blood flow |
| | conduit 259f |
| 56g | Extension of the bottom opening 55g of sample storage well 51g of |
| | measurement cartridge 10g for connecting sample storage well 51g |
| | to manifold 455g |
| 56h | Extension of the bottom opening 55h of sample storage well 51h of |
| | measurement cartridge 10h for connecting sample storage well 51h |
| | to manifold 455h |
| 57a | Sample inlet portion of cartridge 10a, which comprises some |
| | elements of the cartridge that interacts with the cap 200a |
| 57b | Sample inlet portion of cartridge 10b, which comprises some |
| | elements of the cartridge that interacts with the cap 200b |
| 57c | Sample inlet portion of cartridge 10c, which comprises some |
| | elements of the cartridge that interacts with the cap 200c |
| 58d | Sample storage well boss of cartridge 10d for increasing the sample |
| | storage well storage capacity |
| 59a | Flat surface of sample inlet portion 57a |
| 59b | Flat surface of sample inlet portion 57b |
| 59c | Flat surface of sample inlet portion 57c |
| 60a | Second housing member of calibration cartridge 20a |
| 60b | Second housing member of calibration cartridge 20b |
| 61a | Electrochemical sensor array of measurement cartridge 10a having |
| | at least one of an amperometric sensor, a conductivity sensor and a |
| | potentiometric sensor |
| 61b | Electrochemical sensor array of measurement cartridge 10b having |
| | at least one of an amperometric sensor, a conductivity sensor and a |
| | potentiometric sensor |
| 61c | Electrochemical sensor array of measurement cartridge 10c having |
| | at least one of an amperometric sensor, a conductivity sensor and a |
| | potentiometric sensor |
| 61d | Electrochemical sensor array of measurement cartridge 10d having |
| | at least one of an amperometric sensor, a conductivity sensor and a |
| | potentiometric sensor |
| 61f | Electrochemical sensor array of measurement cartridge 10f having at |
| | least one of an amperometric sensor, a conductivity sensor and a |
| | potentiometric sensor |
| 61g | Electrochemical sensor array of measurement cartridge 10g having |
| | at least one of an amperometric sensor, a conductivity sensor and a |
| | potentiometric sensor |
| 61h | Electrochemical sensor array of measurement cartridge 10h having |
| | at least one of an amperometric sensor, a conductivity sensor and a |
| | potentiometric sensor |
| 62a | Electrochemical sensor array of calibration cartridge 20a having at |
| | least one of an amperometric sensor, a conductivity sensor and a |
| | potentiometric sensor |
| 62b | Electrochemical sensor array of calibration cartridge 20b having at |
| | least one of an amperometric sensor, a conductivity sensor and a |
| | potentiometric sensor |
| 64b | Nest for seating directional valve element 69b |
| 65g | Nest for seating directional valve element 67g |
| 66g | Nest for seating directional valve element 68g |
| 66h | Nest for seating directional valve element 68h |
| 67g | First directional valve element of measurement cartridge 10g, which |
| | for example, could be an elastomeric flap |
| 68g | Second directional valve element of measurement cartridge 10g, |
| | which for example, could be an elastomeric flap |
| 68h | Directional valve element of measurement cartridge 10h, which for |
| | example, could be an elastomeric flap |
| 69b | Directional valve element of calibration cartridge 20b, which for |
| | example, may be an elastomeric flap |
| 70 | System for measuring one or more properties of blood, shown in FIG. |
| | 15 |
| 71b | Smaller section of directional valve element 69b that is flappable for |
| | closing off valve seat 327b (see FIG. 8G in conjunction with FIG. 8H) |
| 73b | Larger section of the directional valve element 69b that is used to |
| | seat directional valve element 69b in receptor 64b (see FIG. 8E) |
| 75g | Sealed blister for storing calibration fluid of measurement cartridge |
| | 10g |
| 76g | Compressible blister support for supporting blister 75g over spike |
| | 277g |
| 80 | Analyzer for measuring one or more properties of blood, shown in |
| | FIGS. 18A-18C |
| 81a | Ledge in second housing member 40a of measurement cartridge 10a |
| | for housing electrochemical sensor array 61a |
| 91a | Sealed blister for storing calibration fluid of calibration cartridge 20a |
| 92a | Compressible blister support for supporting sealed blister 91a over |
| | spike 271a |
| 93b | First sealed blister for storing first calibration fluid of calibration |
| | cartridge 20b |
| 95b | Second sealed blister for storing second calibration fluid of calibration |
| | cartridge 20b |
| 96b | Compressible blister support for supporting blister 93b over spike |
| | 273b |
| 97b | Compressible blister support for supporting blister 95b over spike |
| | 275b |
| 99a | Bottom laminate for covering blister outlet conduit 301a |
| 99b | Bottom laminate for covering blister outlet conduits 307b and 309b |
| 99g | Bottom laminate for covering blister outlet conduit 431g |
| 100a | Double-sided sticky gasket of measurement cartridge 10a |
| 100b | Double-sided sticky gasket of measurement cartridge 10b |
| 100c | Double-sided sticky gasket of measurement cartridge 10c |
| 100d | Double-sided sticky gasket of measurement cartridge 10d |
| 100e | Double-sided sticky gasket of measurement cartridge 10e |
| 100f | Double-sided sticky gasket of measurement cartridge 10f |
| 100g | Double-sided sticky gasket of measurement cartridge 10g |
| 100h | Double-sided sticky gasket of measurement cartridge 10h |
| 102a | Double-sided sticky gasket of calibration cartridge 20a |
| 102b | Double-sided sticky gasket of calibration cartridge 20b |
| 103a | Cutout in double-sided sticky gasket 100a aligned with the bottom |
| | opening 55a of sample storage well 51a of cartridge 10a |
| 103b | Cutout in double-sided sticky gasket 100b aligned with the bottom |
| | opening 55b of the sample storage well 51b of cartridge 10b |
| 103e | Cutout in double-sided sticky gasket 100e aligned with the bottom |
| | opening 55e of the sample storage well 51e of cartridge 10e |
| 103f | Cutout in double-sided sticky gasket 100f aligned with the bottom |
| | opening 55f of the sample storage well 51f of cartridge 10f |
| 103g | Cutout in double-sided sticky gasket 100g aligned with the bottom |
| | opening 55g of the sample storage well 51g of cartridge 10g |
| 103h | Cutout in double-sided sticky gasket 100h aligned with the bottom |
| | opening 55h of the sample storage well 51h of cartridge 10h |
| 105a | Cutout in double-sided sticky gasket 100a for mitigating blood flow |
| | from extension 56a of bottom opening 55a during sample loading |
| 105b | Cutout in double-sided sticky gasket 100b for mitigating blood flow |
| | from extension 56b of bottom opening 55b during sample loading |
| 105e | Cutout in double-sided sticky gasket 100e for mitigating blood flow |
| | from extension 56e of bottom opening 55e during sample loading |
| 105f | Cutout in double-sided sticky gasket 100f for mitigating blood flow |
| | from extension 56f of bottom opening 55f |
| 105g | Cutout in double-sided sticky gasket 100g for mitigating blood flow |
| | from extension 56g of bottom opening 55g |
| 105h | Cutout in double-sided sticky gasket 100h for mitigating blood flow |
| | from extension 56h of bottom opening 55h |
| 107a | Cutout in double-sided sticky gasket 100a aligned with hole in sealing |
| | member 241a and corresponding hole 242a in cartridge 10a |
| 109b | Cutout in double-sided sticky gasket 100b aligned with vent 231b of |
| | cartridge 10b |
| 109e | Cutout in double-sided sticky gasket 100f aligned with vent 231e of |
| | cartridge 10e |
| 109f | Cutout in double-sided sticky gasket 100f aligned with vent 231f of |
| | cartridge 10f |
| 113a | Cutout in gasket 100a aligned with blood conduit 259a of |
| | measurement cartridge 10a |
| 113b | Cutout in gasket 100b aligned with blood conduit 259b of |
| | measurement cartridge 10b |
| 115a | Cutout in gasket 102a aligned with electrochemical sensor conduit |
| | 262a of calibration cartridge 20a |
| 115b | Cutout in gasket 102b aligned with electrochemical sensor conduit |
| | 262b of calibration cartridge 20b |
| 116b | Cutout in gasket 100b aligned with electrochemical sensor conduit |
| | 261b of measurement cartridge 10b |
| 117a | Cutout in gasket 102a aligned with blister window 291a of calibration |
| | cartridge 20a |
| 119a | Cutout in gasket 102a aligned with vent 233a of calibration cartridge |
| | 20a |
| 119b | Cutout in gasket 102b aligned with vent 233b of calibration cartridge |
| | 20b |
| 123b | Cutout in gasket 102b aligned with blister window 293b of calibration |
| | cartridge 20b |
| 125b | Cutout in gasket 102b aligned with blister window 295b of calibration |
| | cartridge 20b |
| 127b | Cutout in gasket 102b aligned with transfer conduit 315b of calibration |
| | cartridge 20b |
| 161f | Cutout in gasket 100f aligned with overlap between mixing chambers |
| | 464f and 465f |
| 162f | Cutout in gasket 100f aligned with overlap between mixing chambers |
| | 463f and 464f |
| 163f | Cutout in gasket 100f aligned with overlap between enlarged section |
| | 260f and mixing chamber 463f |
| 165g | Cutout in gasket 100g aligned with inlet 457g of manifold 455g of |
| | measurement cartridge 10g |
| 165h | Cutout in gasket 100h aligned with inlet 457h of manifold 455h of |
| | measurement cartridge 10h |
| 167g | Cutout in gasket 100g, which serves as air bladder communication |
| | port for connecting air bladder duct 421g with smaller section 268g of |
| | second directional valve element 68g |
| 167h | Cutout in gasket 100h, which serves as air bladder communication |
| | port for connecting air bladder duct 421h with smaller section 268h of |
| | second directional valve element 68h |
| 200a | Cap for closing sample inlet portion 57a of measurement cartridge |
| | 10a |
| 200b | Cap for closing inlet portion 57b of measurement cartridge 10b |
| 200c | Cap for closing inlet portion 57c of measurement cartridge 10c |
| 200d | Cap for closing sample storage well 51d of measurement cartridge |
| | 10d |
| 200e | Cap for closing sample storage well 51e of measurement cartridge |
| | 10e |
| 200f | Cap for closing sample storage well 51f of measurement cartridge 10f |
| 200g | Cap for closing sample storage well 51g of measurement cartridge |
| | 10g |
| 200h | Cap for closing sample storage well 51h of measurement cartridge |
| | 10h |
| 203a | Top side of cap 200a |
| 203c | Top side of cap 200c |
| 203e | Top side of cap 200e |
| 203f | Top side of cap 200f |
| 203g | Top side of cap 200g |
| 203h | Top side of cap 200h |
| 205a | Underside of cap 200a, comprising a cap flat surface 211a and a cap |
| | recess 215a |
| 205c | Underside of cap 200c, having a cap flat surface 211c and a cap |
| | recess 215c |
| 205d | Underside of cap 200d, comprising a cap flat surface 211c and a cap |
| | plunger 217d |
| 205e | Underside of cap 200e, comprising a cap flat surface 211e and a cap |
| | plunger 217e |
| 205f | Underside of cap 200f, comprising a cap flat surface 211f and a cap |
| | plunger 217f |
| 205g | Underside of cap 200g, having a cap plunger 217g |
| 205h | Underside of cap 200h, having a cap plunger 217h |
| 208e | Nest in top portion 30e of measurement cartridge 10e for receiving |
| | cap 200e when the cap is in a fully open configuration |
| 209e | Locking slot for capturing cap wing 210e for locking cap 200e in fully |
| | open configuration (2 shown in FIG. 13F) |
| 210e | Cap wing for locking cap 200e in fully open configuration during |
| | loading of sample storage well 51e (2 shown in FIG. 13F) |
| 211a | Cap flat surface disposed at the underside 205a of cap 200a |
| 211c | Cap flat surface disposed at the underside 205c of cap 200c |
| 211d | Cap flat surface disposed at the underside 205d of cap 200d |
| 211e | Cap flat surface disposed at the underside 205e of cap 200e |
| 211f | Cap flat surface disposed at the underside 205f of cap 200f |
| 215a | Cap recess in the underside 205a of cap 200a |
| 215b | Cap recess in the underside of cap 200b |
| 215c | Cap recess in the underside 205c of cap 200c |
| 217d | Cap plunger of cap 200d |
| 217e | Cap plunger of cap 200e |
| 217f | Cap plunger of cap 200f |
| 217g | Cap plunger of cap 200g |
| 218e | Overflow trough of sample storage well 51e |
| 218f | Overflow trough of sample storage well 51f |
| 218g | Overflow trough of sample storage well 51g |
| 219e | Overflow groove of sample storage well 51e (4 shown as an example) |
| 219f | Overflow groove of sample storage well 51f (4 shown as an example) |
| 220e | Cap plunger seal of cap plunger 217e, e.g., a rubber O-ring or a |
| | molded O-ring |
| 220f | Cap plunger seal of cap plunger 217f, e.g., a rubber O-ring or a |
| | molded O-ring |
| 220g | Cap plunger seal of cap plunger 217g, e.g., a rubber O-ring or a |
| | molded O-ring |
| 221c | Gasket for cap 200c for turning cap recess 215c into a sealed |
| | chamber when the cap is in a closed configuration |
| 231b | Cartridge vent of measurement cartridge 10b |
| 231c | Cartridge vent of measurement cartridge 10c |
| 231d | Cartridge vent of measurement cartridge 10d |
| 231e | Cartridge vent of measurement cartridge 10e |
| 231f | Cartridge vent of measurement cartridge 10f |
| 231g | Cartridge vent of measurement cartridge 10g |
| 231h | Cartridge vent of measurement cartridge 10h |
| 232a | Hinge for hingedly attaching cap 200a to body of cartridge 10a |
| 232d | Hinge for hingedly attaching cap 200d to body of cartridge 10d |
| 232e | Hinge for hingedly attaching cap 200e to body of cartridge 10e |
| 233a | Cartridge vent of calibration cartridge 20a |
| 233b | Cartridge vent of calibration cartridge 20b |
| 235a | Cap latch for engaging cap 200a to body of cartridge 10a |
| 235d | Cap latch for engaging cap 200d to body of cartridge 10d |
| 236a | Cap latch catch in body of cartridge 10a for engaging cap latch 235a |
| 236d | Cap latch catch in body of cartridge 10d for engaging cap latch 235d |
| 241a | Sealing member installed in nest 243a in measurement cartridge 10a, |
| | for frictionally engaging an analyzer pump probe, which may be a flat |
| | surface or a ball having a channel for estblishing connection between |
| | an associated analyzer pump and waste receptacle 255a |
| 241c | Sealing member installed in cartridge air inlet duct 247c in |
| | measurement cartridge 10c, for frictionally engaging the outer |
| | surface of an associated analyzer pump hollow needle |
| 242a | Hole in first housing member 30a of measurement cartridge 10a, |
| | aligned with hole in sealing member 241a |
| 243a | Nest for sealing member 241a |
| 247c | Cartridge duct for housing sealing member 241c |
| 253a | Cap vent in cartridge cap 200a of cartridge 10a |
| 255a | Waste receptacle of measurement cartridge 10a |
| 256a | Waste receptacle of calibration cartridge 20a |
| 256b | Waste receptacle of calibration cartridge 20b |
| 258b | Waste receptacle of measurement cartridge 10b |
| 258c | Waste receptacle of measurement cartridge 10c |
| 258d | Waste receptacle of measurement cartridge 10d |
| 258e | Waste receptacle of measurement cartridge 10e |
| 258f | Waste receptacle of measurement cartridge 10f |
| 258g | Waste receptacle of measurement cartridge 10g |
| 259a | Blood conduit for fluidly connecting sample storage well 51a to |
| | detection chamber 261a |
| 259b | Blood conduit for fluidly connecting sample storage well 51b to |
| | detection chamber 412b (an optical chamber) |
| 259c | Blood conduit for fluidly connecting sample storage well 51c to |
| | detection chamber 261c |
| 259d | Blood conduit for fluidly connecting sample storage well 51d to |
| | detection chamber 261d |
| 259e | Blood conduit for fluidly connecting sample storage well 51e to |
| | detection chamber (in this cartridge the detection chamber is optical |
| | chamber 412e) |
| 259f | Blood conduit for fluidly connecting sample storage well 51f to optical |
| | chamber 412f and electrochemical sensor chamber 261f |
| 260a | Enlarged section of blood conduit 259a for minimizing, mitigating, or |
| | modifying blood flow from extension 56a of bottom opening 55a of |
| | sample storage well 51a during sample loading |
| 260e | Enlarged section of blood conduit 259e for minimizing, mitigating, or |
| | modifying blood flow from extension 56e of bottom opening 55e of |
| | sample storage well 51e during sample loading |
| 260f | Enlarged section of blood conduit 259f for minimizing, mitigating, or |
| | modifying blood flow from extension 56f of bottom opening 55f of |
| | sample storage well 51f |
| 260g | Enlarged section for minimizing, mitigating, or modifying blood flow |
| | from extension 56g of bottom opening 55g of sample storage well |
| | 51g, and for fluidly connecting cutouts 105g and 165g of gasket 100g |
| 260h | Enlarged section for minimizing, mitigating, or modifying blood flow |
| | from extension 56h of bottom opening 55h of sample storage well |
| | 51h, and for fluidly connecting cutouts 105h and 165h of gasket 100h |
| 261a | Detection chamber (in this cartridge it is a biosensor chamber or an |
| | electrochemical sensor chamber) of measurement cartridge 10a |
| 261b | Biosensor or an electrochemical sensor chamber of measurement |
| | cartridge 10b |
| 261c | Detection chamber (in this cartridge it is a biosensor or an |
| | electrochemical sensor chamber) of measurement cartridge 10c |
| 261d | Detection chamber (in this cartridge it is a biosensor or an |
| | electrochemical sensor chamber) of measurement cartridge 10d |
| 261f | Electrochemical sensor chamber of measurement cartridge 10f |
| 261g | Electrochemical sensor chamber of measurement cartridge 10g |
| 262a | Electrochemical sensor chamber/conduit of calibration cartridge 20a |
| 262b | Electrochemical sensor chamber/conduit of calibration cartridge 20b |
| 264g | Larger section of first directional valve element 67g |
| 265g | Larger section of second directional valve element 68g |
| 267g | Smaller section of first directional valve element 67g |
| 268g | Smaller section of second directional valve element 68g |
| 271a | Spike for rupturing sealed blister 91a |
| 273b | Spike for rupturing the sealed blister 93b |
| 275b | Spike for rupturing the sealed blister 95b |
| 277g | Spike for rupturing the sealed blister 75g |
| 279g | Through hole in spike 277g for draining calibration fluid from ruptured |
| | blister 75g |
| 291a | Blister window in the first housing member 50a of calibration cartridge |
| | 20a for accessing sealed blister 91a |
| 292a | Through hole in spike 271a for draining calibration fluid from ruptured |
| | blister 91a |
| 293b | Blister window in the first housing member 50b of calibration cartridge |
| | 20b for accessing sealed blister 93b |
| 295b | Blister window in the first housing member 50b of calibration cartridge |
| | 20b for accessing sealed blister 95b |
| 296b | Through hole in spike 273b for draining calibration fluid from ruptured |
| | blister 93b |
| 297b | Through hole in spike 275b for draining calibration fluid from ruptured |
| | blister 95b |
| 298g | Blister window in the first housing member 30g of measurement |
| | cartridge 10g for accessing the sealed blister 75g |
| 301a | Calibration liquid conduit for receiving calibration liquid from blister |
| | 91a after the calibration liquid is released |
| 302a | Transfer conduit for transferring calibration fluid from conduit 301a to |
| | conduit 303a |
| 303a | Pre-electrochemical sensor conduit for receiving calibration fluid from |
| | transfer conduit 302a and delivering calibration fluid to |
| | electrochemical sensor conduit 262a |
| 303b | Pre-electrochemical sensor conduit for receiving calibration fluid from |
| | either transfer conduit 311b (from blister 93b) or transfer conduit 317b |
| | (from blister 95b), and delivering each calibration fluid to |
| | electrochemical sensor conduit 262b at different times |
| 305a | Post-electrochemical sensor conduit for receiving excess calibration |
| | fluid from electrochemical sensor conduit 262a |
| 305b | Post-electrochemical sensor conduit for receiving excess calibration |
| | fluid from electrochemical sensor conduit 262b |
| 307b | Blister outlet conduit for receiving calibration fluid from the ruptured |
| | blister 93b |
| 309b | Blister outlet conduit for receiving calibration fluid from the ruptured |
| | blister 95b |
| 311b | Transfer conduit for transferring calibration fluid from conduit 307b to |
| | conduit 303b |
| 315b | Transfer conduit for transferring calibration fluid from conduit 309b to |
| | transfer conduit 317b |
| 317b | Transfer conduit for transferring calibration fluid from transfer conduit |
| | 315b to conduit 303b |
| 327b | Valve seat for mating with smaller section 71b of directional valve |
| | element 69b (see FIG. 8G in conjunction with FIG. 8H) |
| 331g | Valve seat for mating with smaller section 267g of directional valve |
| | element 67g |
| 333g | Valve seat for mating with smaller section 268g of directional valve |
| | element 68g |
| 401g | Blood conduit for fluidly connecting sample storage well 51g to optical |
| | chamber 412 |
| 401h | Blood conduit for fluidly connecting sample storage well 51h to optical |
| | chamber 412h |
| 402g | Blood conduit for fluidly connecting sample storage well 51g to |
| | electrochemical sensor chamber 261g |
| 402h | Blood conduit for fluidly connecting sample storage well 51h to |
| | electrochemical sensor chamber 261h |
| 403b | Pre-electrochemical sensor conduit in measurement cartridge 10b |
| 403g | Pre-electrochemical sensor conduit in measurement cartridge 10g |
| 403h | Pre-electrochemical sensor conduit in measurement cartridge 10h |
| 405g | Post-electrochemical sensor conduit in measurement cartridge 10g |
| 405h | Post-electrochemical sensor conduit in measurement cartridge 10h |
| 411b | First optical window of optical chamber 412b |
| 411e | First optical window of optical chamber 412e |
| 411f | First optical window of optical chamber 412f |
| 411g | First optical window of optical chamber 412g |
| 411h | First optical window of optical chamber 412h |
| 412b | Optical chamber of measurement cartridge 10b (may be a gasket |
| | cutout if the gasket thickness provides sufficient optical pathlength) |
| 412e | Optical chamber of measurement cartridge 10e (may be a gasket |
| | cutout if the gasket thickness provides sufficient optical pathlength) |
| 412f | Optical chamber of measurement cartridge 10f (may be a gasket |
| | cutout if the gasket thickness provides sufficient optical pathlength) |
| 412g | Optical chamber of measurement cartridge 10g (may be a gasket |
| | cutout if the gasket thickness provides sufficient optical pathlength) |
| 412h | Optical chamber of measurement cartridge 10h |
| 413b | Second optical window of optical chamber 412b |
| 413e | Second optical window of optical chamber 412e |
| 413f | Second optical window of optical chamber 412f |
| 413g | Second optical window of optical chamber 412g |
| 413h | Second optical window of optical chamber 412h |
| 417b | Air bladder of cartridge 10b |
| 417f | Air bladder of cartridge 10f |
| 417g | Air bladder of cartridge 10g |
| 417h | Air bladder of cartridge 10h |
| 419b | Air bladder laminate of air bladder 417b of cartridge 10b |
| 419f | Air bladder laminate of air bladder 417f of cartridge 10f |
| 419g | Air bladder laminate of air bladder 417g of cartridge 10g |
| 419h | Air bladder laminate of air bladder 417h of cartridge 10h |
| 421b | Air bladder duct for providing fluid connection between an air bladder |
| | 417b and an air bladder communication port 423b |
| 421f | Air bladder duct for providing fluid connection between an air bladder |
| | 417f and an air bladder communication port 163f |
| 421g | Air bladder duct for providing fluid connection between an air bladder |
| | 417g and an air bladder communication port 167g |
| 421h | Air bladder duct for providing fluid connection between an air bladder |
| | 417h and an air bladder communication port 167h |
| 423b | Air bladder communication port of a sample inlet portion 57b of |
| | cartridge 10b |
| 423c | Associated analyzer pump communication port of sample inlet |
| | portion 57c of cartridge 10c |
| 427b | One of one or more female cartridge tracks for guiding linear motion |
| | of cap 200b. In this non-limiting example, two female tracks are |
| | shown. In some embodiments, the one or more tracks may be |
| | configured as male cartridge tracks. Some embodiments may |
| | comprise one male and one female track, and if desired, the cap |
| | motion may be non-linear (i.e. curved). |
| 431g | Blister outlet conduit for receiving calibration fluid from the ruptured |
| | blister 75g |
| 433g | Transfer conduit for transferring calibration fluid from conduit 431g to |
| | pre-electrochemical sensor conduit 403g |
| 435g | Conduit for connecting conduit 402g to conduit 403g |
| 451c | Hydrophobic insert disposed close to the bottom opening 55c of the |
| | sample storage well 51c, for providing means for minimizing, |
| | mitigating, or modifying blood flow out of the sample storage well 51c |
| 453c | Nest in second housing member 40c of cartridge 10c for installing |
| | hydrophobic insert 451c |
| 455g | Manifold of extension 56g of the bottom opening 55g of sample |
| | storage well 51g of cartridge 10g, having an inlet 457g |
| 455h | Manifold of extension 56h of the bottom opening 55h of sample |
| | storage well 51h of cartridge 10h |
| 457g | Inlet of manifold 455g |
| 457h | Inlet of manifold 455h |
| 463f | First mixing chamber of measurement cartridge 10f |
| 464f | Second mixing chamber of measurement cartridge 10f |
| 465f | Third mixing chamber of measurement cartridge 10f |
| 467b | Blood shunt in measurement cartridge 10b |
| 467f | Blood shunt in measurement cartridge 10f |
| 470h | Overlap between blood conduit 402h and pre-electrochemical sensor |
| | conduit 403h of measurement cartridge 10h |

### Overview of Calibration Cartridges 20a and 20b as Non-Limiting Examples

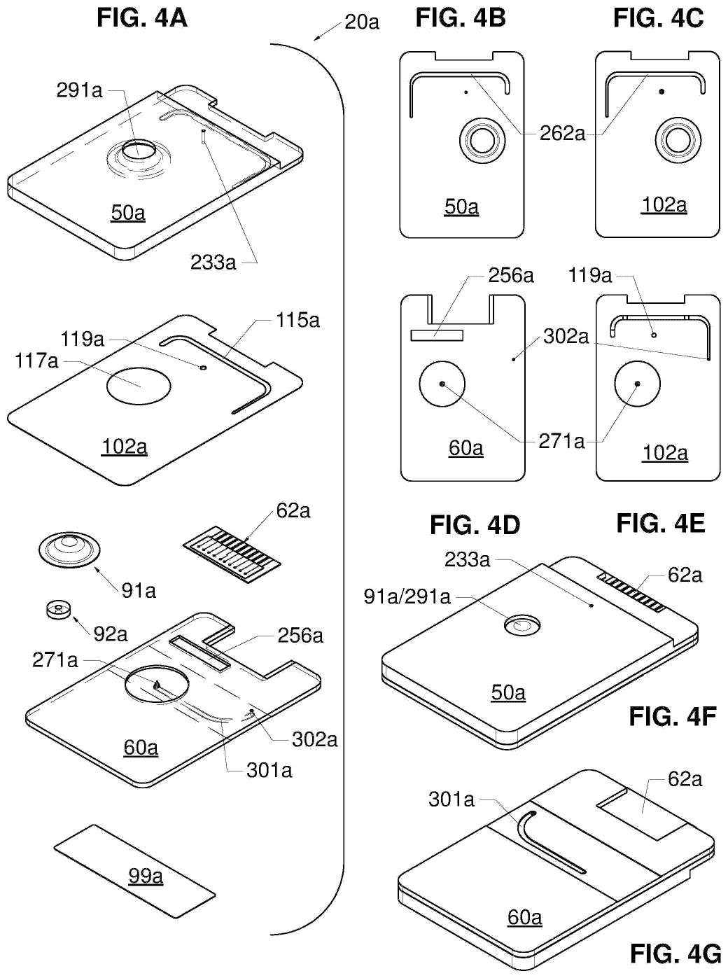

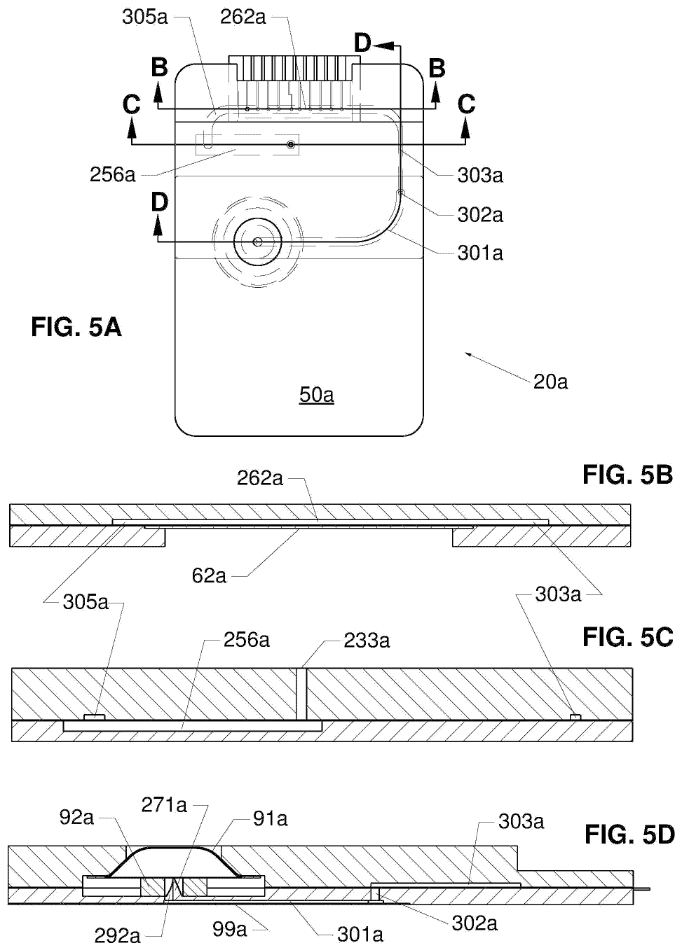

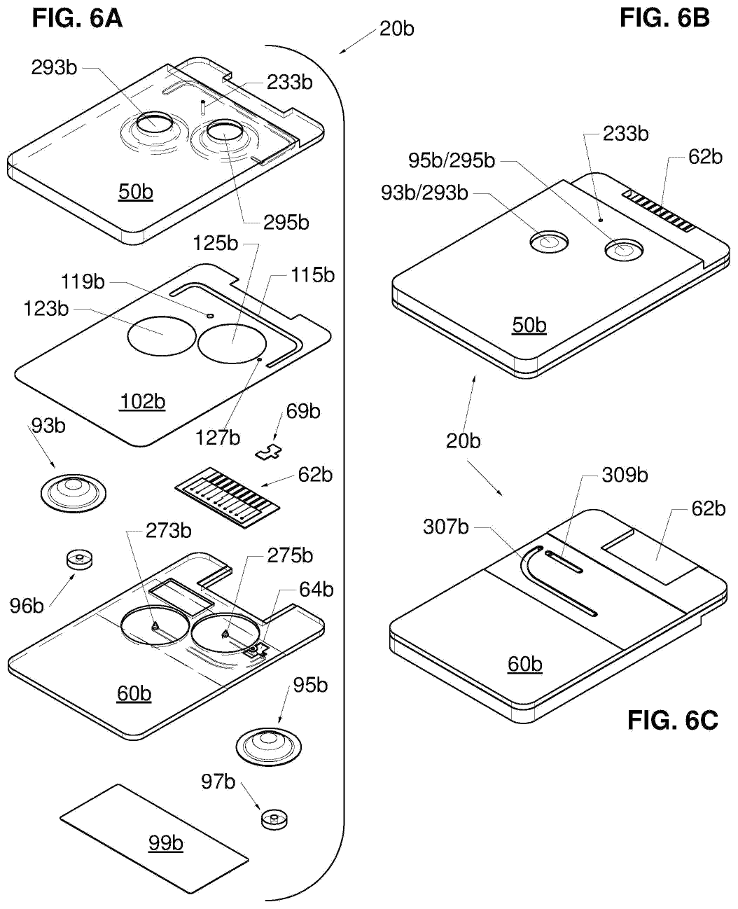

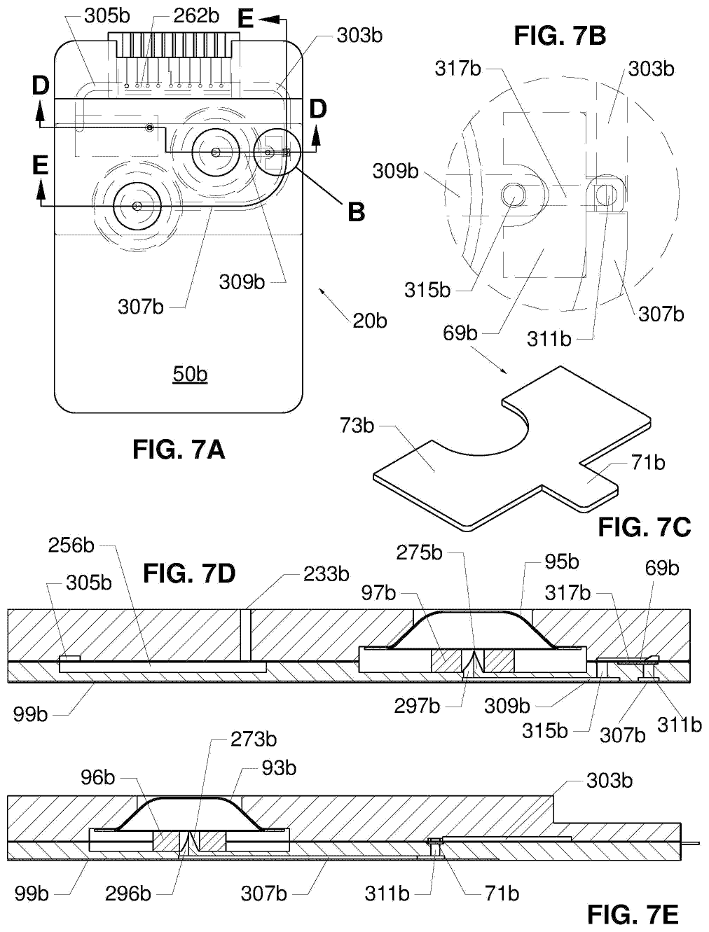

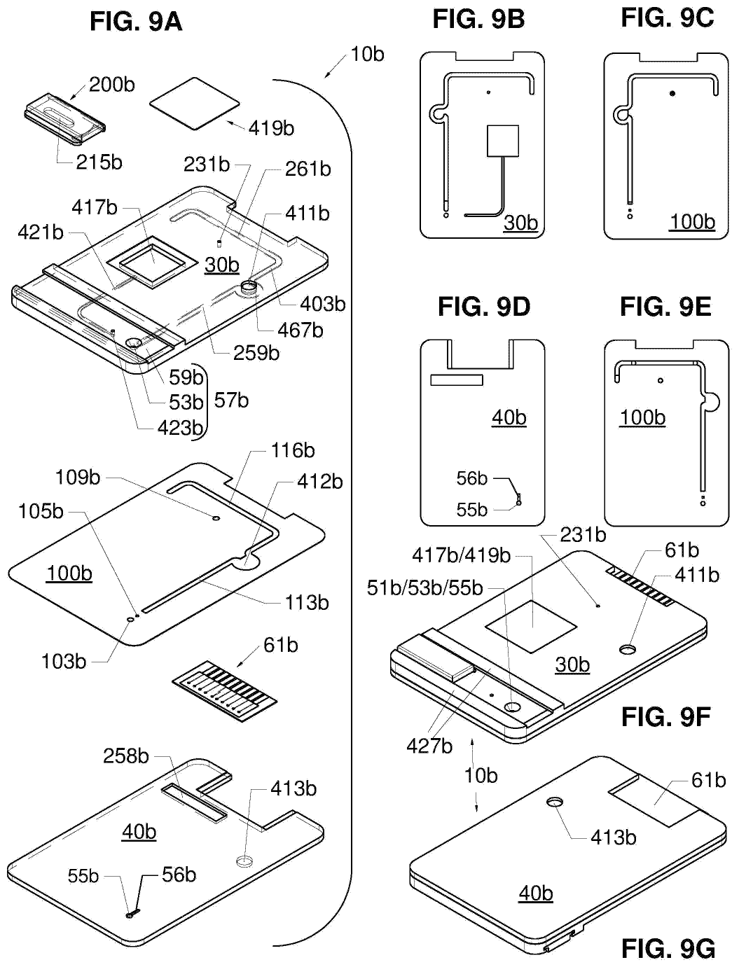

Two embodiments of calibration cartridges are provided: Calibration cartridge 20a is illustrated collectively in FIGS. 4A-5D, and calibration cartridge 20b is illustrated collectively in FIGS. 6A-8H. Description of the structural features is provided in Table 1. The major difference between the two calibration cartridges is that calibration cartridge 20a comprises a single calibration liquid blister 91a, illustrated in FIG. 4A, an exploded view of the calibration cartridge, and FIG. 5D, an enlarged cross-sectional view of the calibration cartridge along lines D-D shown in FIG. 5A. Calibration cartridge 20a may be used for a single-point calibration. Similar cartridges may also be used for monitoring quality control of the associated analyzer, since the quantities of the analytes are known. In contrast, calibration cartridge 20b comprises two sealed calibration liquid blisters 93b and 95b, illustrated in FIG. 6A, an exploded view of the calibration cartridge, and FIGS. 7D and 7E, enlarged cross-sectional views of the calibration cartridge along lines D-D and E-E respectively, shown in FIG. 7A. Calibration cartridge 20b, which comprises an electrochemical sensor array 62b (see FIGS. 6A-6C) may be used to perform two-point calibration to calibrate electrochemical sensor array 61b (see FIGS. 9A, 9F and 9G) installed in measurement cartridge 10b. In this example of a measurement cartridge 10b, the electrochemical sensor array 61b is similar to the electrochemical sensor array 62b installed in calibration cartridge 20b, and preferably belong to the same manufactured batch.

As an alternative to a calibration cartridge comprising two sealed calibration liquid blisters for performing two-point calibration, two calibration cartridges each comprising a single calibration liquid blister may be used, wherein each of the two calibration liquid blisters in the two calibration cartridges are located in the same position, and the liquid composition of the two calibration liquid blisters are different. An advantage to this alternative is that the analyzer only requires a single rupture mechanism. A rupture mechanism may be a stepper motor actuator, as an example, which pushes against the blister, and the same actuator may also be used to activate an air bladder, if the cartridge comprises an air bladder. Further, multi-point calibration may be performed using more than two calibration cartridges, each calibration cartridge comprising a single calibration liquid blister, wherein the single calibration liquid blisters in the more than two calibration cartridges are located in the same position, and the liquid composition of each of the single calibration liquid blisters is different. When more than one calibration cartridges, each having a single calibration blister, the calibration liquid in each calibration cartridge is released and tested sequentially.

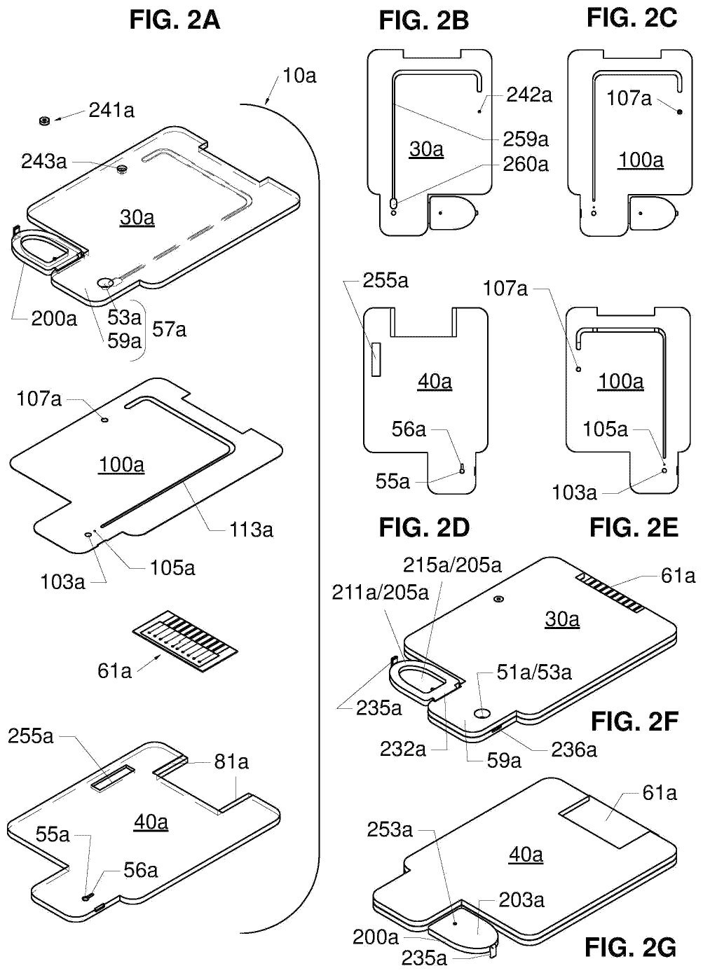

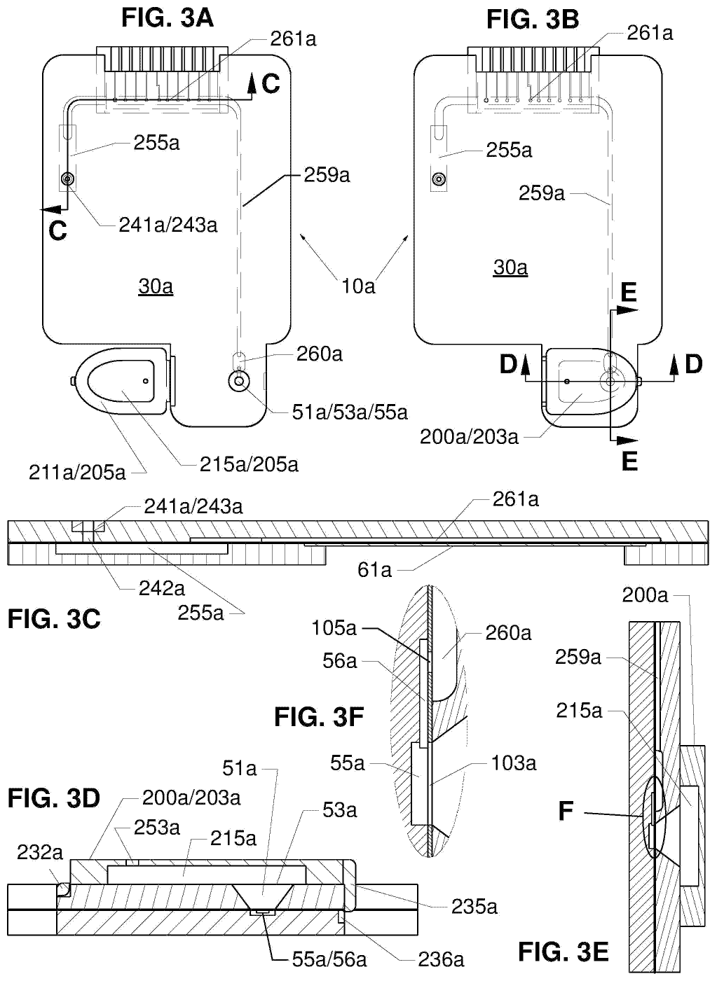

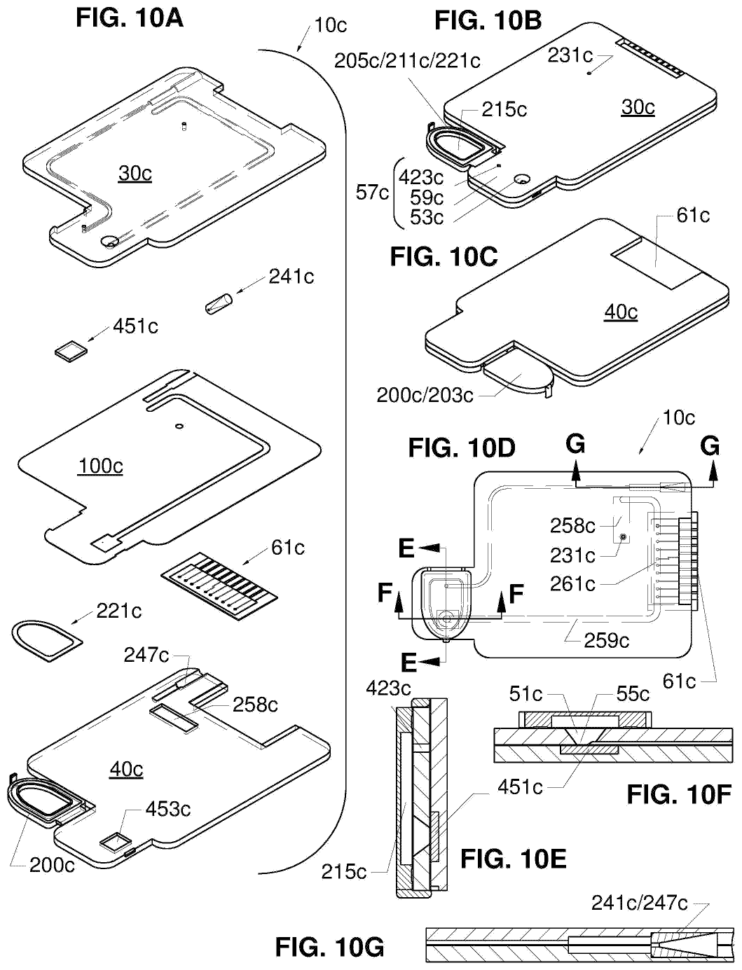

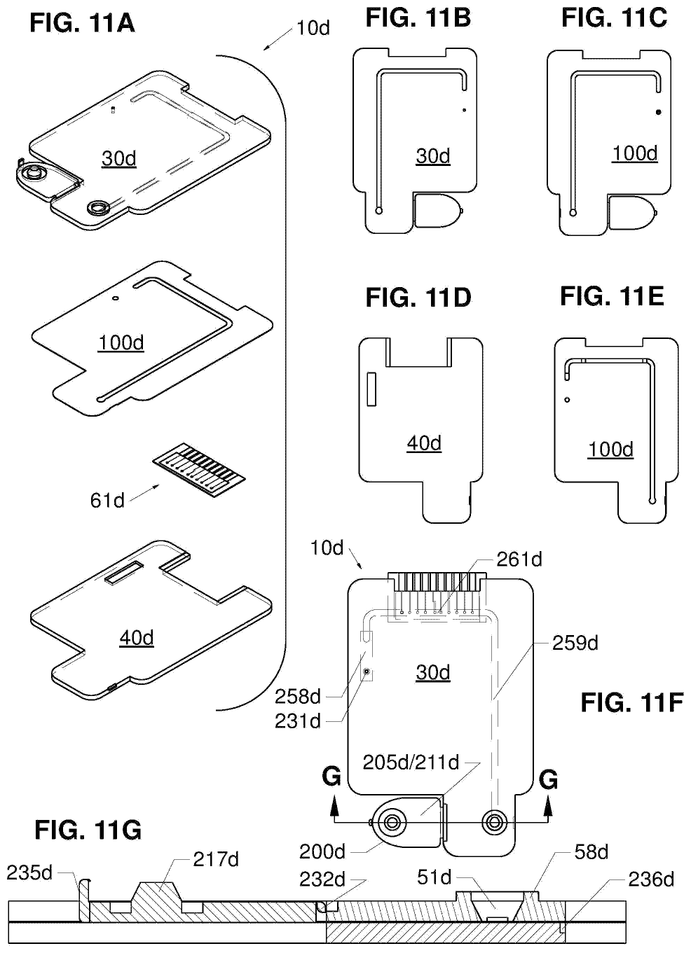

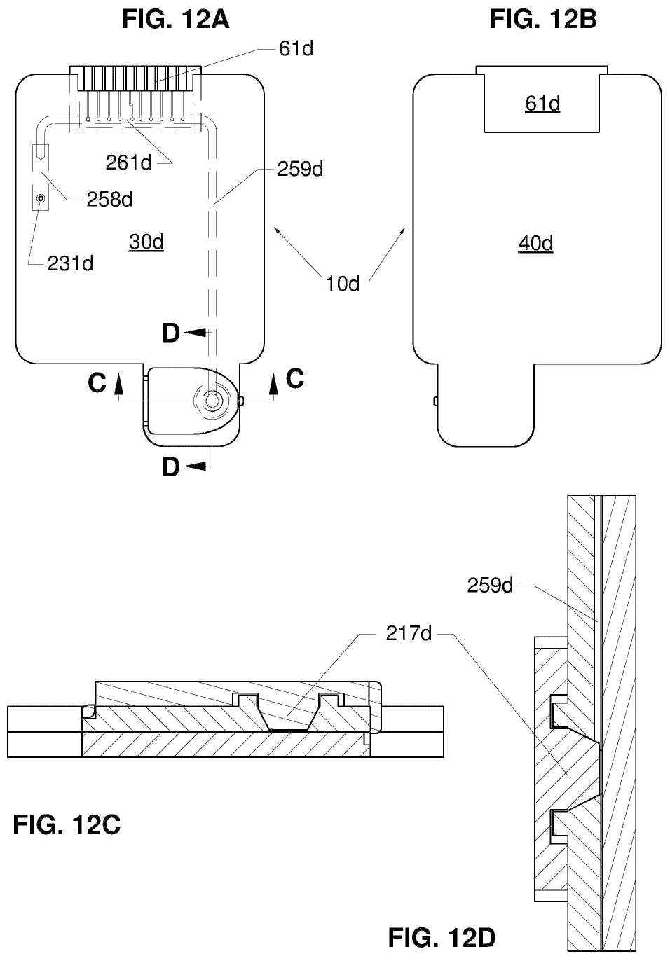

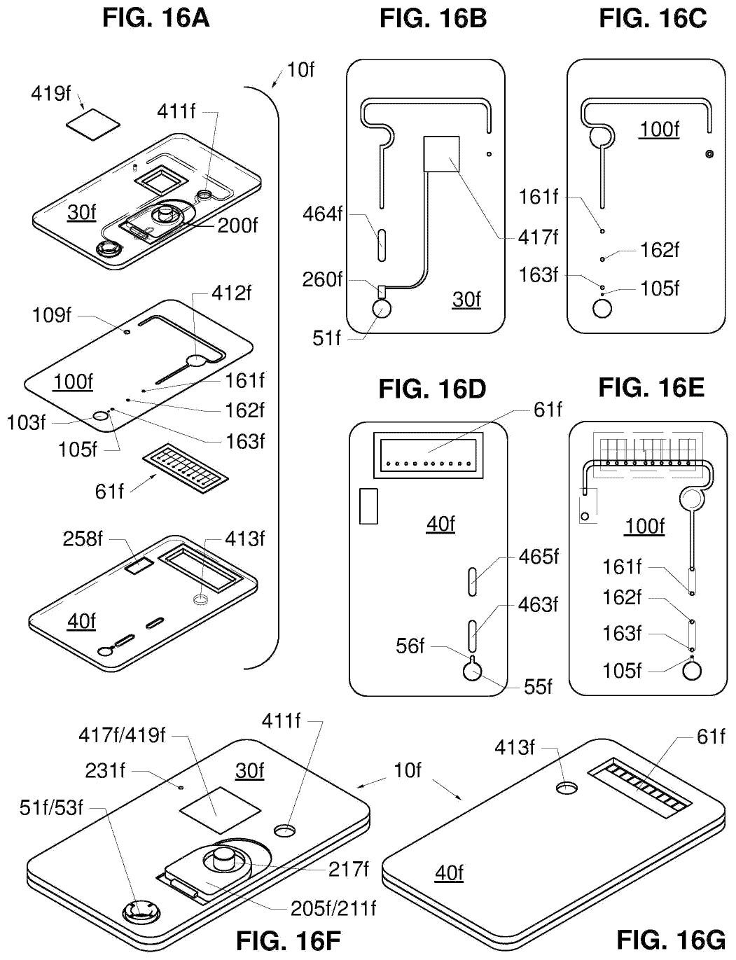

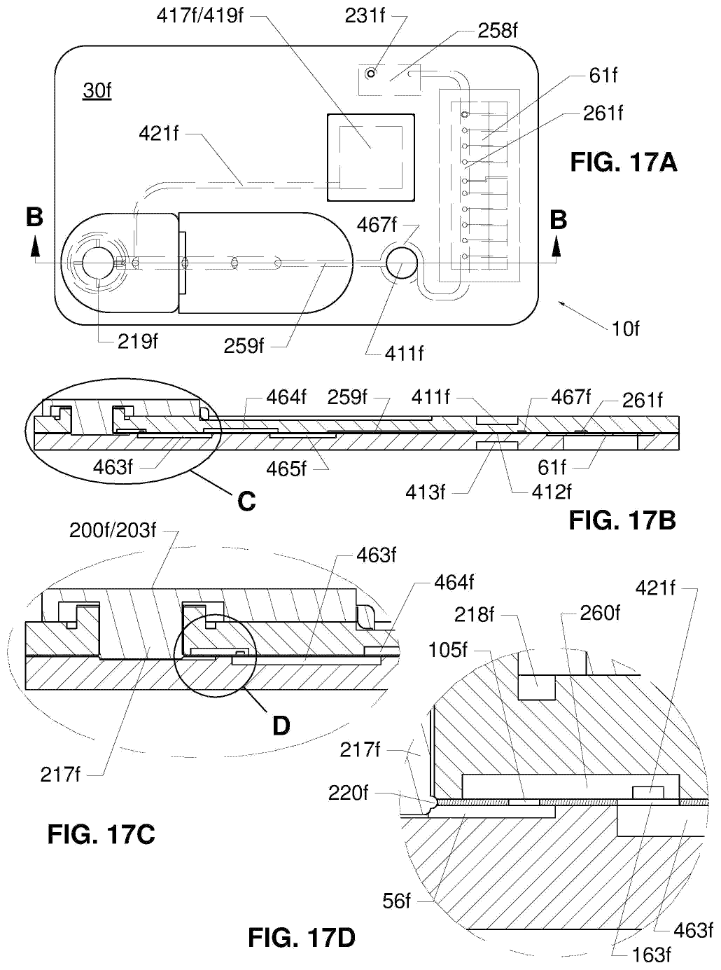

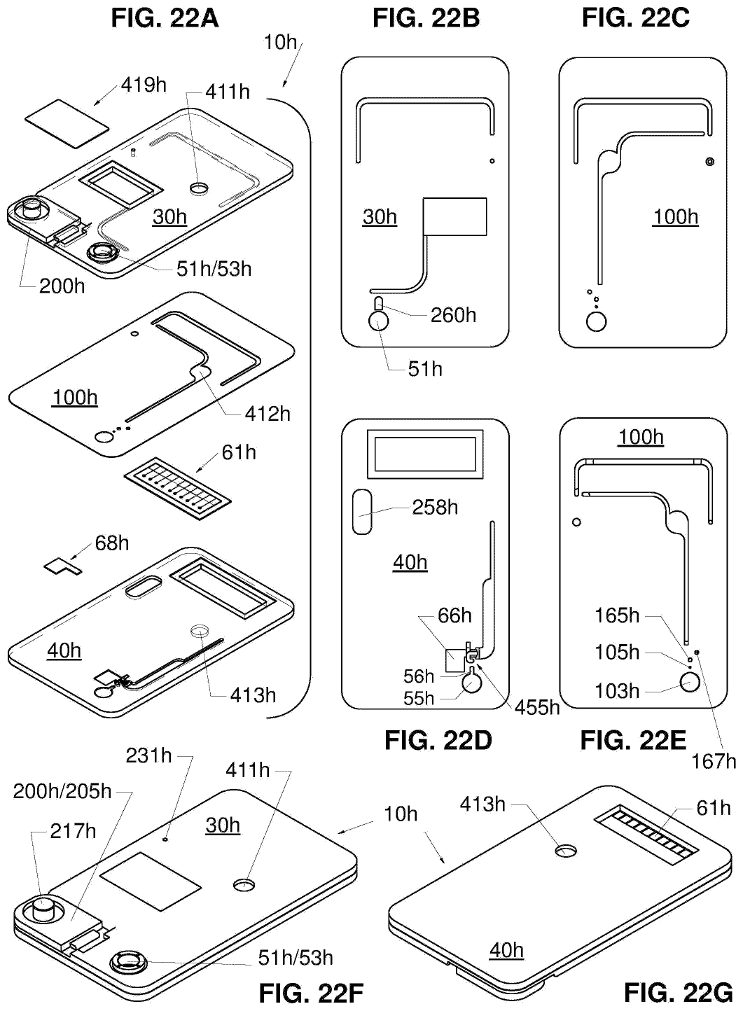

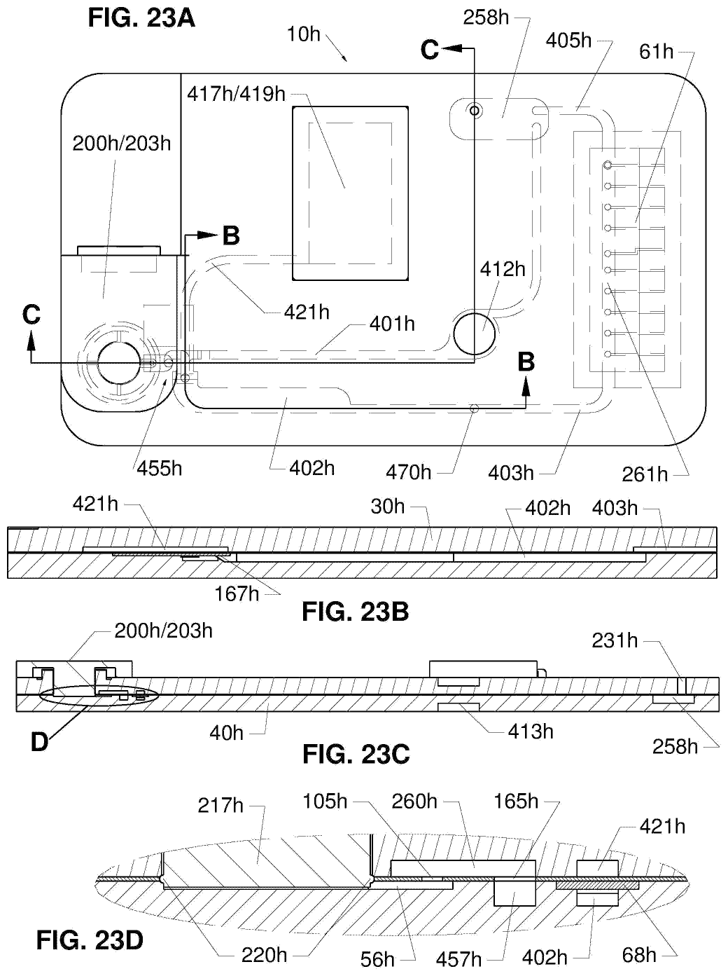

Other measurement cartridges that may be calibrated with calibration cartridges 20a or 20b include measurement cartridge 10a (shown in FIGS. 2A-3E), measurement cartridge 10c (shown in FIGS. 10A-10G), measurement cartridge 10d (shown in FIGS. 11A-12D), measurement cartridge 10f (shown in FIGS. 16A-17D), and measurement cartridge 10h (shown in FIGS. 22A-23D). Neither of these cartridges include a calibration liquid blister, and they all contain electrochemical sensor arrays 61a, 61c, 61d, 61f, and 61h respectively. Calibration cartridge 20b may be used to perform periodic two-point calibration of measurement cartridge 10g; each measurement cartridge 10g is capable of performing one-point calibration because measurement cartridge 10g comprises one sealed blister 75g.

Calibration cartridge 20b, measurement cartridge 10b and analyzer 80 are used as examples to illustrate a system shown in FIGS. 18A-18C. FIG. 18A is a perspective top view of an analyzer 80 and the calibration cartridge 20b, not yet inserted in the receptor 14 of analyzer 80. FIG. 18B is a perspective top view of the analyzer 80 shown in FIG. 18A and the measurement cartridge 10b, not yet inserted in the receptor 14 of analyzer 80. FIG. 18C is a perspective top view of the analyzer 80 and the measurement cartridge 10b shown in FIG. 18B, with the cartridge inserted in the receptor 14 of the analyzer 80 for sample measurement. Prior to insertion of the measurement cartridge 10b, calibration cartridges 20a or 20b comprising electrochemical sensor arrays 62a and 62b respectively, and may be used to calibrate one or more electrochemical sensors of electrochemical sensor array 61b of measurement cartridge 10b illustrated collectively in FIGS. 9A-9G.

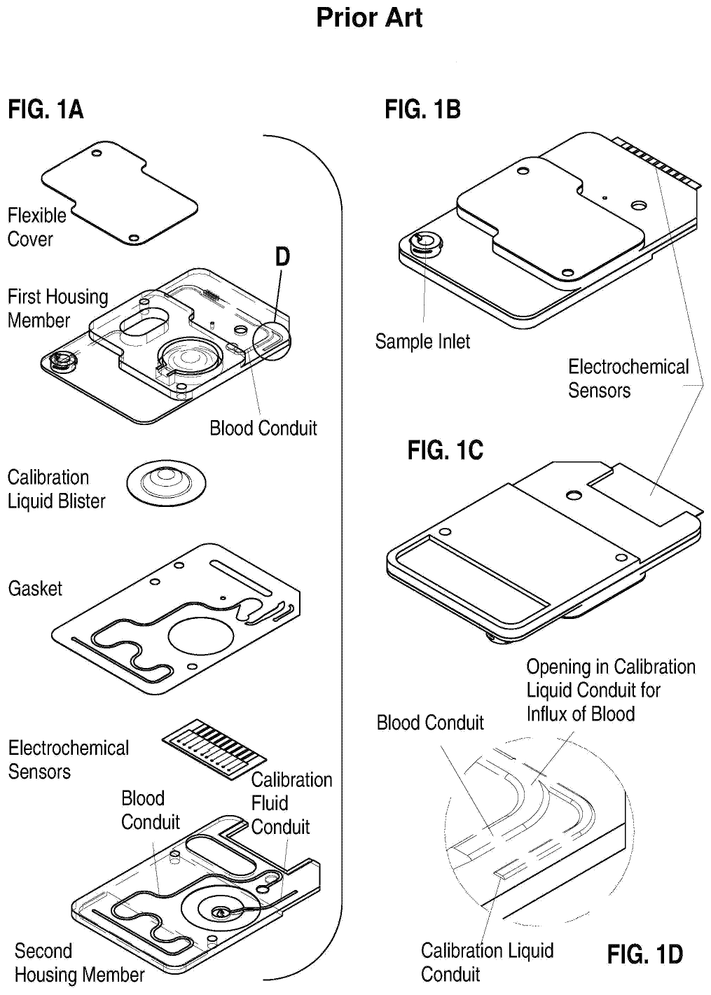

Calibration of one or more electrochemical sensors in electrochemical sensor array 61b of measurement cartridge 10b, using calibration cartridge 20a is described: Force from an attachment to a stepper motor, as a non-limiting example, in an associated analyzer is applied to the top portion (dome portion) of the blister 91a via blister window 291a (see FIG. 4A), pushing the bottom portion (flat portion) of the blister against spike 271a and simultaneously compressing compressible blister support 92a (see FIG. 5D). The spike 271a ruptures the blister releasing calibration liquid into calibration liquid conduit 301a via through hole 292a in spike 271a. Conduit 301a is exposed in FIG. 4G by removing laminate 99a. In other embodiments, for example the prior art shown in FIG. 1A, the spike does not have a through hole, and the calibration liquid flows towards a hole in the gasket and makes its way to the electrochemical sensors, and such flow is considered to be within the scope of the present application. In the prior art, the calibration liquid merges with the blood conduit as shown in FIG. 1D. Referring to FIG. 5D, calibration liquid is transferred from conduit 301a to pre-electrochemical sensor conduit 303a via transfer conduit 302a. Excess calibration liquid leaving the electrochemical sensor conduit 262a (see FIG. 5A) enters conduit 305a and subsequently into a waste receptacle 256a. Cartridge vent 233a (see FIG. 5C) provides an air escape route.

Although calibration cartridges 20a and 20b are both shown to comprise first housing members 50a and 50b attached to second housing members 60a and 60b by double-sided sticky gaskets 102a and 102b respectively, calibration cartridges comprising different housing members in terms of design and number of components are considered to be within the scope of the present application.

Calibration cartridge 20b shown collectively in FIGS. 6A-8H, functions in a similar manner to calibration cartridge 20a, and the calibration liquid blisters are ruptured at different times in order to generate two separate set of electrical signals corresponding to the analyte concentrations. Some embodiments do not include optional directional valve element 69b, which allows either blister to be ruptured first, provided that the associated analyzer is programmed to direct which blister is ruptured first. In this example, the directional valve element may be a flappable polymeric element having a larger section 73b for constraining element 69b, and a smaller section 71b that is flappable to seal off a first conduit while the liquid flows through the second conduit. For example, as illustrated in FIG. 7D, when liquid from blister 95b flows through conduits 317b via conduits 309b and 315b in that order, the flap 71b closes off conduit 311b, which is in fluid communication with blister 93b. On the other hand, when liquid flows through conduit 311b from blister 93b via conduit 307b, the flap 71b is pushed upwards and closes off conduit 317b as the flap 71b is pushed against valve seat 327 shown in FIG. 8G. Operation of directional valve element 69b is illustrated collectively in FIGS. 8A-8H, in conjunction with the description of structural features provided in Table 1. Although no more than two blisters are illustrated in the drawings, any number of blisters are considered to be within the scope of calibration cartridges. An air bubble automatically inserted between the two different calibration liquids may be used to keep the liquids separate, and the air bubble is also effective in removing residues of the first calibration liquid, as the second calibration liquid flows over the electrochemical sensor array.

### Overview of Measurement Cartridges 10a, 10b and 10c as Non-Limiting Examples

A first embodiment of a measurement cartridge 10a is illustrated collectively in FIGS. 2A-3E. Description of the structural features is provided in Table 1. Measurement cartridge 10a comprises an electrochemical sensor array 61a that is similar to electrochemical sensor arrays 62a and 62b in calibration cartridges 20a and 20b respectively. Unlike the calibration cartridges, measurement cartridges are designed to receive a blood sample for measurement. Measurement cartridge 10a is illustrated as a first housing member 30a attached to a second housing member 40a by a double-sided sticky gasket 100a, and comprises a hinged cap 200a, adjustable from a first position to a second position. In the first position, illustrated in FIGS. 2F and 3A, the sample storage well 51a is configured to receive a blood sample via top opening 53a. In the second position, the hinged cap 200a is closed over sample storage well 51a. Hinged cap 200a comprises a cap recess 215a disposed at the underside 205a of cap 200a, and a cap vent 253a. Gravity allows the blood to flow to the bottom opening 55a, and depending on the wettability or hydrophilicity of the material lining the sample storage well 51a and the extension 56a of bottom opening 55a of sample storage well 51a, blood may flow up to cutout 105a in gasket 100a. Due to the small size of gasket cutout 105a and relatively large size of enlarged section 260a of blood conduit 259a (see FIG. 3F), blood flow out of gasket cutout 105a is mitigated, except when the blood is subjected to negative pressure, via sealing member 241a installed in nest 243a in measurement cartridge 10a, for frictionally engaging an analyzer pump probe. Instead of depending on gravity to pull a drop of blood into the sample storage well from a pinprick of the skin of a patient, the bottom of the sample storage well may be corona treated to make the bottom surface more wettable. It was observed that when the bottom of the sample storage well is hydrophobic, the blood tends to cling to the skin until the drop of blood becomes large enough, allowing the force of gravity to overcome the attraction between the blood and the patient's skin. A person skilled in the art would understand that there are alternatives to corona treatment for making a surface hydrophilic, for example, overmolding the bottom of the sample storage well with a hydrophilic plastic by injection molding. Overmolding is a less expensive technique in injection molding than installing a sample storage well insert made of hydrophilic material, which would be an extra step in the manufacturing process. An example of a sample storage well insert is 441j illustrated in FIG. 22A of U.S. Pat. No. 10,661,270 to Samsoondar. As another alternative, at least the second housing member of the measurement cartridge may be injection molded using a hydrophilic plastic like, for example, PETG (polyethylene terephthalate glycol).

The pump probe may be a flat surface or a ball having a channel for establishing connection between an associated analyzer pump and waste receptacle 255a. After the sample storage well 51a receives blood sample, hinged cap 200a is moved from the first position to the second position shown in FIG. 3B. Cap latch 235a and catch 236a keeps the cartridge in the closed configuration, and the cartridge in the closed configuration is placed in an associated analyzer receptor, for example receptor 14 in analyzer 80 illustrated in FIGS. 18A-18C. Analyzers may comprise receptors that swing out or slide out, and after the cartridge is placed in the receptor, it swings in or slides in. In the associated analyzer, a sealing member 241a installed in nest 243a in measurement cartridge 10a (see FIG. 3C), frictionally engages with a pump probe from the associated analyzer. After the analyzer pump is activated, the sample is sucked into the detection chamber 261a via a blood conduit 259a. Any excess blood is trapped in the waste receptacle 255a. Cap vent 253a exposes the blood in the sample storage well to atmospheric pressure, for facilitating blood flow.

Blood conduit in cartridge 10a is shown as the combination of a groove 259a in the first housing member 30a and a cutout 113a in gasket 100a, but in order to minimize sample requirement, the blood conduit may only be the gasket cutout 113a, for example 259e shown in FIG. 13C regarding cartridge 10e.

A third embodiment of a measurement cartridge 10c is illustrated collectively in FIGS. 10A-10G. Compared with measurement cartridge 10a discussed previously, the blood flow mechanism in measurement cartridge 10c is reversed. This is accomplished by replacing the cap vent 253a shown in FIG. 3D with a cartridge vent 231c shown in FIGS. 10B and 10D, and setting the associated analyzer pump to exert positive pressure. In the closed configuration, cap recess 215c creates a closed chamber and air pressure from the associated analyzer pump, via pump communication port 423c (see FIGS. 10E and 10F). In this example, sealing member 241c installed in cartridge air inlet duct 247c in measurement cartridge 10c (see FIG. 10G) is frictionally engaged with the outer surface of an associated analyzer pump hollow needle, which is another example of pump engagement. Another difference in measurement cartridge is the inclusion of a hydrophobic insert 451c disposed close to the bottom opening 55c of the sample storage well 51c, for providing means for minimizing, mitigating, or modifying blood flow out of the sample storage well 51c. The hydrophobic insert 451c located in a nest 453c in the second housing member 40c is illustrated in FIGS. 10E and 10F, viewed in conjunction with FIG. 10D.

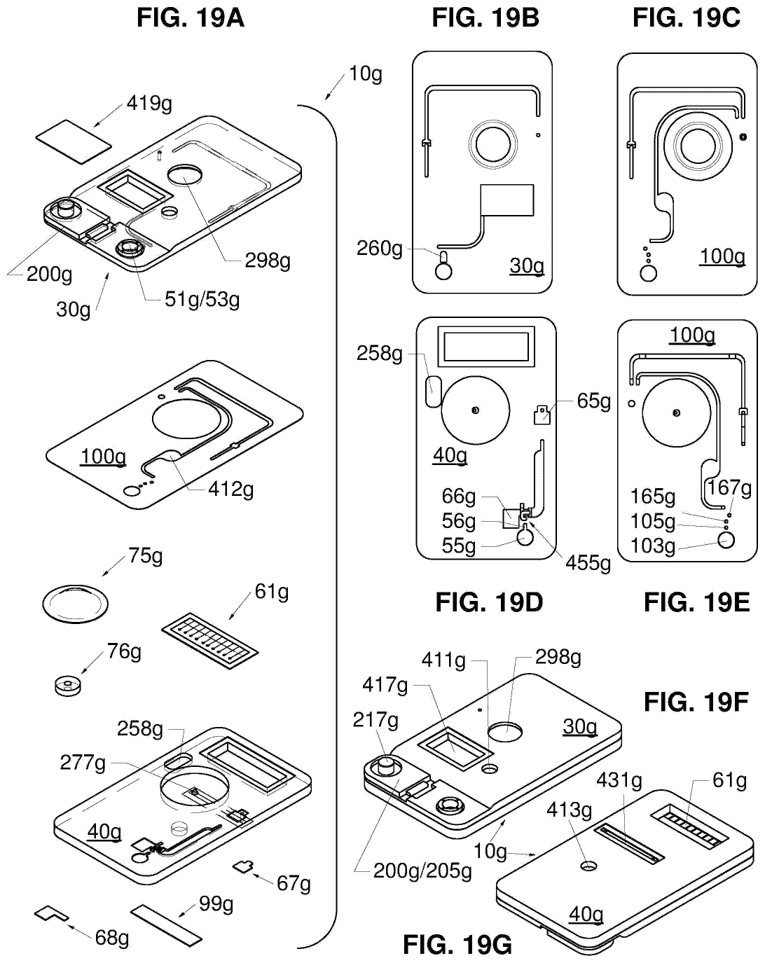

A second embodiment of a measurement cartridge 10b is illustrated collectively in FIGS. 9A-9G. Compared with measurement cartridge 10c discussed previously, the positive pressure used to push the blood sample from the top portion 53b of the sample storage well 51b is not from an associated analyzer pump but instead is generated from an air bladder 417b, illustrated in FIGS. 9A and 9F. A second difference is that instead of a hinged cap, the cap 200b slides along tracks 427b, illustrated in FIG. 9F. The sliding cap 200b also comprises a recess 215b and a sample inlet portion 57b, illustrated in FIG. 9A. A third difference is the inclusion of an optical chamber 412b, enclosed by a first optical window 411b and a second optical window 413b. Although the optical chamber is located between the sample storage well 51b and the electrochemical sensor chamber 261b, the optical chamber may be located downstream of the electrochemical sensor chamber 261b. Moreover, instead of having the two detection chambers (optical and electrochemical sensor) arranged in series, they may also be arranged in parallel, for example, see measurement cartridge 10g illustrated collectively in FIGS. 19A-21J and measurement cartridge 10h illustrated collectively in FIGS. 22A-23D.

Measurement cartridges like 10a, 10b and 10c are discussed in PCT/CA2020/051254 filed Sep. 18, 2020. Other relevant cartridges discussed in PCT/CA2020/051254 include measurement cartridges that slide about a pivotal hinge instead of sliding along tracks.

### Overview of Measurement Cartridges 10d and 10e as Non-Limiting Examples

A fourth embodiment of a measurement cartridge 10d is illustrated collectively in FIGS. 11A-12D. Description of the structural features is provided in Table 1. The hinged cap 205d in measurement cartridge 10d comprises a cap plunger 217d, illustrated in FIG. 11G and viewed in conjunction with FIG. 11F, with the hinged cap 205d in a first position and the sample storage well 51d in an open configuration. Illustrated in FIGS. 12C and 12D, viewed in conjunction with FIG. 12A, the hinged cap 205d is adjusted to second position, wherein the sample storage well is in a closed configuration. In the open configuration, the sample storage well 51d is configured to receive a blood sample. Depending on the hydrophobicity of the blood conduit 259d, some blood may or may not flow from the sample storage well 51d into the blood conduit 259d. If desirable, means for minimizing, mitigating, or modifying blood flow out of the sample storage well 51d, as described for measurement cartridges 10a and 10c may be included in the design of measurement cartridge 10d. During the time when the hinged cap 205d is moved from the first position to the second position, the cap plunger displaces blood from the sample storage well 51d into the detection chamber 261d via a blood conduit 259d. Air pressure in the detection chamber 261d is relieved by cartridge vent 231d. Any excess blood is contained in the waste receptacle 258d. In cartridge 10d, neither air pressure (positive or negative) nor capillary action is required to move blood from the sample storage well 51d to the detection chamber 261d. The advantages of a measurement cartridge having a cap comprising a plunger cap like 217d are: 1) Simpler less expensive measurement cartridge; 2) More options in plastics used for manufacture of measurement cartridge; and 3) Simpler less expensive associated analyzer. A person skilled in the art of injection molding should understand that the plunger cap may be an overmolding element made from a different material. For example, the plunger and O-ring may be overmolded as a single element using a thermoplastic elastomer (TPE), and the rest of the cartridge may be constructed using a harder and more transparent plastic, for example polyethylene terephthalate (PET) or glycol modified PET (i.e. PETG). As another example, the entire plunger cap, including the O-ring may be made from TPE as an overmolding feature.

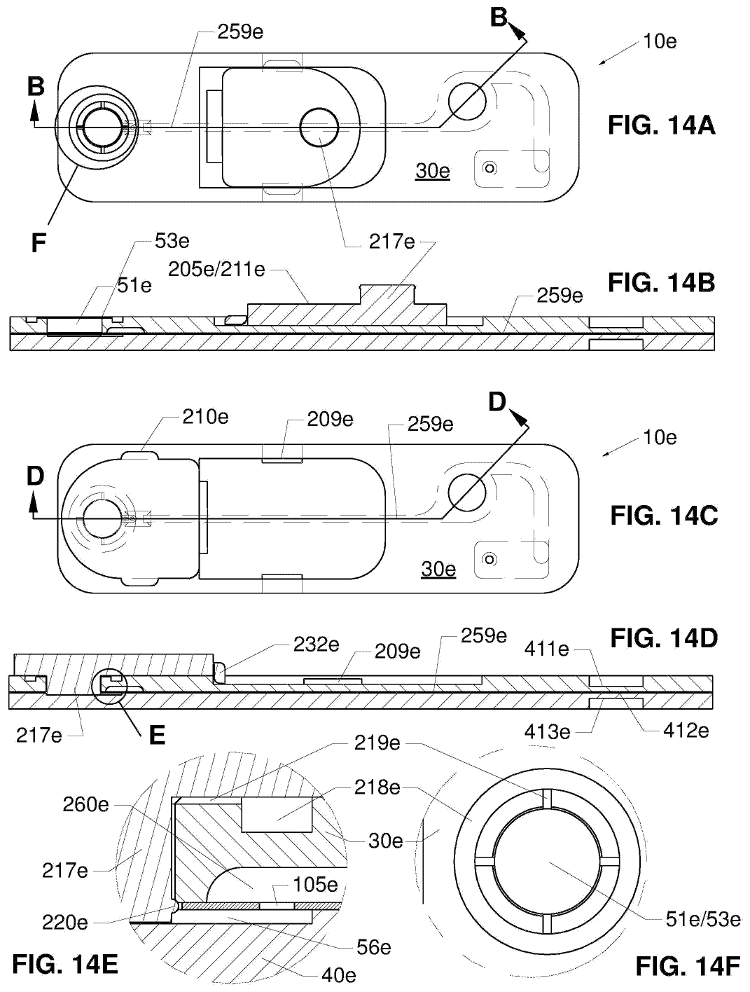

A fifth embodiment of a measurement cartridge 10e illustrated collectively in FIGS. 13A-14F is similar to cartridge 10d. A first difference is that the plunger 217e illustrated in FIG. 14D, viewed in conjunction with FIGS. 14C and 14E, is cylindrical comprising an O-ring 220e. The O-ring may be a rubber slip-on O-ring or plastic, molded as an integral part of the plunger 217e. A second difference is that the detection chamber is an optical chamber 412e enclosed by a first optical window 411e and a second optical window 413e. A third difference is the inclusion of an enlarged section 260e of blood conduit 259e for minimizing, mitigating, or modifying blood flow from extension 56e of bottom opening 55e of sample storage well 51e during sample loading, as was described for measurement cartridge 10a. A fourth difference is the inclusion of overflow groove 219e of sample storage well 51e (4 shown as an example), and an overflow trough 218e of sample storage well 51e for containing any excess blood. After the cartridge is adjusted from an open configuration to a closed configuration, the O-ring remains located in the groove at the gasket, preventing the plunger from rebounding. With the overflow grooves 219e and the enlarged section 260e, gasket cutout 105e, the volume of blood displaced by the plunger 217e is substantially reproducible from cartridge to cartridge. The reproducibility of the volume of blood displaced by the plunger 217e also depends on the wettability of the sample storage well surface and grooves 219e of the sample storage well. Some embodiments of cartridge body constructed from hydrophobic material may comprise a sample storage well as an insert, wherein the insert is constructed from a more hydrophilic or wettable material than the rest of the cartridge body. If the surfaces of the sample storage well is too hydrophobic, the blood sample may not fill the sample storage well completely, and the overflow groves 219e may not function properly, producing a bulging meniscus of the blood sample in the well. As an alternative to the enlarged section 260e and gasket cutout 105e, a hydrophobic insert (e.g., 451c in FIGS. 10E and 10F) may be installed at the outlet 55e of the sample storage well 51e, as illustrated in FIG. 10F of cartridge 10c.

The sample storage capacity of the sample storage well 51e may be altered by changing the diameter of the well 51e. The sample storage capacity of the sample storage well 51e may also be altered without changing the diameter of the well 51e, by increasing or decreasing the depth of the well 51e. As shown in FIG. 14D, the top of the sample storage well is aligned with the top surface of the first housing member 30e, and as shown in FIG. 17B regarding cartridge 10f, the top of the sample storage well is above the top surface of the first housing member 30f. The top of the sample storage well may also be below the top surface of the first housing member of a measurement cartridge. In order to reduce dead volume, the length of the plunger 217e is sufficiently long to reach the bottom of the sample storage well 51f. In order to avoid crushing red blood cells, a small space is maintained between the bottom of the plunger 217e and the bottom of the sample storage well 51e, by designing the cap 200e so that the cap flat surface 211e makes contact with the top surface of the first housing member 30e when the cap 200e is adjusted from the first position to the second position.

### Overview of Measurement Cartridges 10f as a Non-Limiting Example

A sixth embodiment of a measurement cartridge 10f is illustrated collectively in FIGS. 16A-17D. Description of the structural features is provided in Table 1. Shown in FIG. 16A is an exploded perspective top view of the measurement cartridge 10f for measuring at least one property of blood, comprising a first housing member 30f, a second housing member 40f, and a double-sided sticky gasket 100f for attaching housing members 30f and 40f. Shown in FIG. 16B is a bottom view of the first housing member 30f of the cartridge shown in FIG. 16A, and shown in FIG. 16C is the bottom view of the first housing member 30f of the cartridge shown in FIG. 16B, overlaid by and in alignment with a gasket 100f shown in FIG. 16A. Shown in FIG. 16D is a top view of the second housing member 40f of the cartridge shown in FIG. 16A, and shown in FIG. 16E is the top view of the second housing member 40f shown in FIG. 16D, overlaid by and in alignment with the gasket 100f shown in FIG. 16A.

FIG. 16F illustrates a perspective top view of the cartridge 10f in the assembled state, showing the upper surface of the cartridge body, with cap 200f adjusted to a first position, whereby the sample storage well 51f is in an open configuration for receiving a blood sample. FIG. 16G illustrates a perspective bottom view of the cartridge 10f showing the lower surface of the cartridge body. After receiving the blood sample, the cap is adjusted from the first position to a second position as shown in FIG. 17A, whereby the sample storage well 51f is in a closed configuration. The O-ring 220f remains located in the groove at the gasket, preventing the plunger from rebounding. Although the O-ring groove is shown to be at the gasket interface with the first housing member 30f and second housing member 40f, the groove may be at other locations, and the position of the O-ring adjusted in a corresponding manner. With overflow grooves 219f, enlarged section 260f, and gasket cutout 105f (see FIG. 17D), the volume of blood displaced by the plunger 217f is substantially reproducible from cartridge to cartridge. When the cap 200f is adjusted from a first position to a second position, a metered volume of blood is displaced from the sample storage well 51f into a mixing chamber 463f (see FIGS. 16D and 17C), which may contain predetermined amounts of one or more dry reagents, for example without any limitations, a hemolyzing agent. Turbulence further mixes the metered volume of blood and the predetermined amount(s) of reagent(s) as the mixture or altered blood is moved from the mixing chamber 463f to mixing chamber 464f, to mixing chamber 465f, into the blood conduit 259f, and finally into the detection chambers 412f (optical) and 261f (electrochemical). Cartridge 10f comprises both an optical chamber 412f enclosed by a first optical window 411f and a second optical window 413f, and an electrochemical sensor chamber 261f (see FIGS. 17A and 17B). Some measurement cartridges do not include a mixing chamber and may contain one or more dry reagents in any section of the blood flow conduit between the top portion of the sample storage well and the detection chamber, and the means for mixing the blood and the one or more dry reagents includes the one or more reagents, blood flow, and dissolution of the one or more reagents when the blood flows over the one or more reagents.

Movement of altered blood from the mixing chamber 463f is facilitated by pressurized air from air bladder 417f via air bladder duct 421f and air bladder communication port 163f. Therefore, movement of unaltered blood and movement of altered blood are two separate steps, utilizing the plunger 217f and the air bladder 417f respectively. Optional use of an associated analyzer pump instead of an air bladder 417f was previously discussed.

Illustrated in FIG. 17B is an enlarged cross-sectional view through the measurement cartridge 10f shown in FIG. 17A along line B-B. Shown in FIG. 17C is a detailed view of detail C shown in FIG. 17B, and shown in FIG. 17D is a detailed view of detail D shown in FIG. 17C.

### Overview of Measurement Cartridges 10g and 10h as Non-Limiting Examples

A seventh embodiment of a measurement cartridge 10g is illustrated collectively in FIGS. 19A-21J, and an eighth embodiment of a measurement cartridge 10h is illustrated collectively in FIGS. 22A-23D, for measuring at least one property of blood. Description of the structural features is provided in Table 1. Measurement cartridge 10g is very similar to measurement cartridge 10h; a major difference is that cartridge 10g comprises a calibration fluid blister 75g for performing a 1-point calibration (i.e., offset correction).

Shown in FIG. 19A is an exploded perspective top view of the measurement cartridge 10g. With the parts of cartridge 10g assembled, shown in FIG. 19F is a perspective top view of the cartridge 10g shown in FIG. 19A, with cap 200g adjusted to a first position, wherein the sample storage well 51g is configured to receive a blood sample. Shown in FIG. 19G is a perspective bottom view of the cartridge 10g shown in FIG. 19A. The separate first housing member 30g, second housing member 40g and their interaction with double-sided sticky gasket 100g used to hold 30h and 40h together are illustrated in FIGS. 19B-19E: Shown in FIG. 19B is a bottom view of the first housing member 30g of the cartridge shown in FIG. 19A; shown in FIG. 19C is the bottom view of the first housing member 30g of the cartridge shown in FIG. 19B, overlaid by and in alignment with the gasket 100g shown in FIG. 19A; shown in FIG. 19D is a top view of the second housing member 40g of the cartridge shown in FIG. 19A; and shown in FIG. 19E is the top view of the second housing member 40g shown in FIG. 19D, overlaid by and in alignment with the gasket 100g shown in FIG. 19A. Similar illustrations for measurement cartridge 10h are provided in FIGS. 22A-22G.

Some structural features and views are illustrated for either measurement cartridge 10g or 10h and not in both. Therefore, in order to understand the cartridges functionality, references may be made to structural features and views for either measurement cartridge 10g or 10h, and the cartridges are recognized by the letters “g” and “h” respectively. After blood is placed in the sample storage well 51g shown in FIG. 19A, gravity allows the blood to fall to the bottom 55g (see FIG. 19D) of sample storage well 51g. With reference to FIG. 23D, blood flow may stop at cutout 105h in double-sided sticky gasket 100h due to the relatively small area of cutout 105h fluidly connected to an enlarged section 260h. Another option for providing means for minimizing, mitigating, or modifying blood flow out of the sample storage well 51h is illustrated in FIGS. 10E and 10F regarding measurement cartridge 10c, wherein the means for minimizing, mitigating, or modifying blood flow out of the sample storage well 51c includes hydrophobic insert 451c disposed close to the bottom opening 55c of the sample storage well 51c. After the sample storage well 51g receives a blood sample, with cap 200g in a first position, the blood sample is advanced in a first stage and a second stage, which is discussed next.

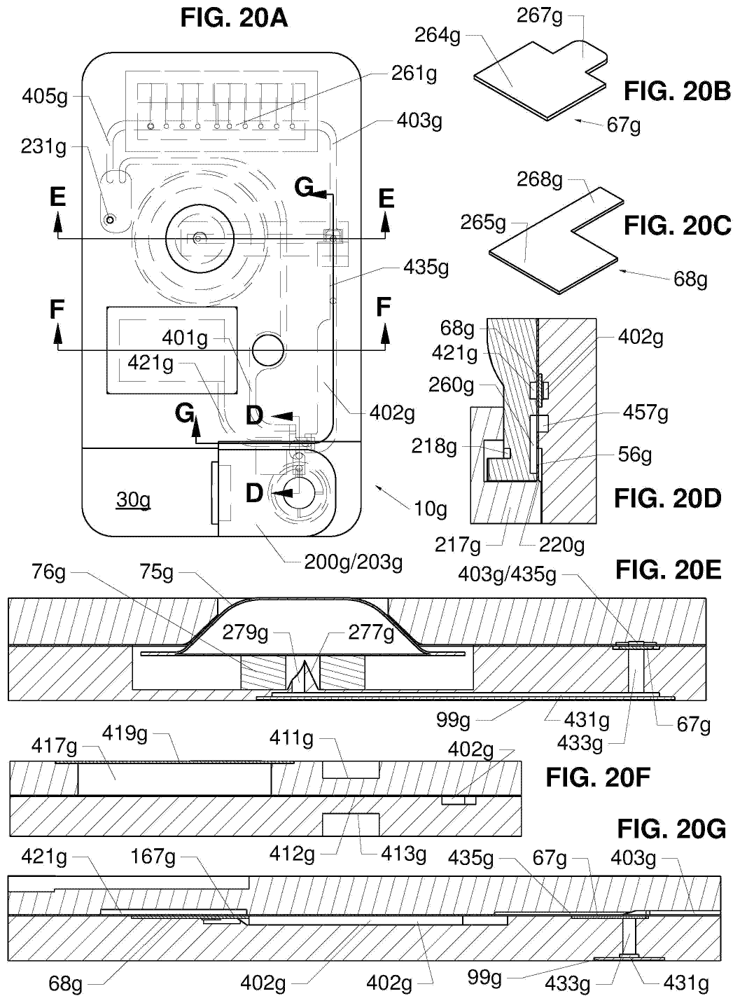

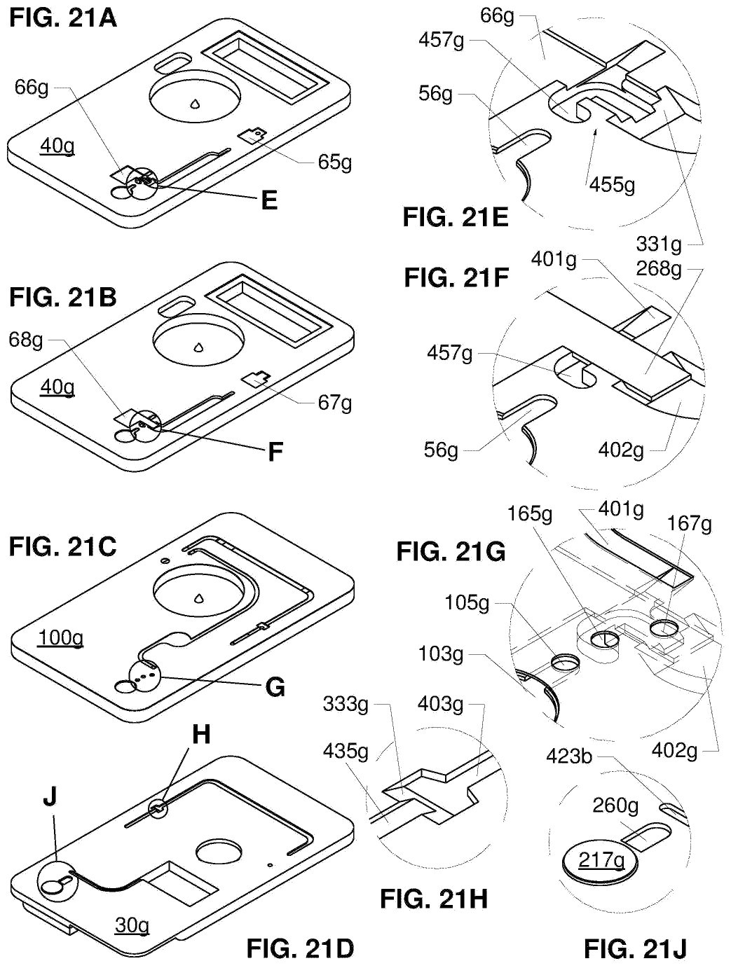

In the first stage, cap 200g is adjusted from the first position to a second position, wherein in the second position the cartridge is configured so that the plunger 217g in cap 200g displaces at least some of the blood in sample storage well 51g through bottom opening 55g. The displaced blood flows through manifold 455g (see FIGS. 19D and 21E) via gasket cutout 105h illustrated in FIG. 21G, viewed in conjunction with FIGS. 23A and 23D regarding measurement cartridge 10h. Regarding measurement cartridge 10g (see FIG. 20A), manifold 455g splits the blood flow into blood conduits 401g and 402g. Blood conduit 401g is sufficiently small to allow blood to fill optical chamber 412g and allow some excess blood to flow towards waste receptacle 258g. The depth of the optical chamber is relatively shallow: preferably about 50-200 microliters. Due to the larger size of blood conduit 402g, a larger volume of blood enters blood conduit 402g. In the second configuration of measurement cartridge 10g, plunger 21g by design, pushes blood into blood conduit 402g, but not into electrochemical sensor chamber 261g until after the sensors in electrochemical sensor array 61g are calibrated (one-point) with calibration liquid from blister 75g. After calibration liquid is released from blister 75g and forced into electrochemical sensor chamber 261g for calibrating the sensors, blood from blood conduit 402g displaces the calibration liquid and the electrical signals from the blood is collected after the blood comes in contact with the sensors. Preventing blood flow into the electrochemical sensor chamber 261h of measurement cartridge 10h directly from the manifold 455h is not a requirement, because no sensor calibration is performed. However, an advantage to the two-step blood flow provides the benefit of using a smaller blood volume. Blood in the optical chamber 412g or 412h may be interrogated with electromagnetic radiation (EMR) any time after optical chamber 412g or 412h is filled with altered or unaltered blood. Altered blood is a mixture of blood and one or more reagents, for example a hemolyzing agent. In some applications, it may be beneficial to hemolyze only the blood entering the optical chamber 412h because hemolyzed blood scatters less EMR, therefore more EMR is transmitted through the blood sample providing stronger signals for the analyte of interest. On the other hand, hemolyzed blood is not desirable for measuring certain plasma analytes, for example potassium, because the concentration of potassium inside the red blood cells is about 20 times higher than the potassium concentration in plasma.

In the second stage, positive air pressure from, for example, an air bladder 417h pushes the blood in blood conduit 402h into electrochemical sensor chamber 261h for measurement by the one or more sensors in electrochemical sensors array 61h. Other means for pushing blood into electrochemical sensor chamber 261h includes an associated analyzer pump, as described regarding measurement cartridge 10c illustrated collectively in FIGS. 10A-10G. The pressurized air from air bladder 417h via air bladder duct 421h can only enter blood conduit 402h and cannot enter blood conduit 401h. This feature is illustrated in FIGS. 21E-21G regarding measurement cartridge 10g, viewed in conjunction with FIGS. 23B-23D regarding measurement cartridge 10h: Smaller section 268g of directional valve element 68g (68h regarding measurement cartridge 10h) folds against valve seat 331g, under pressurized air from air bladder duct 421g (see FIGS. 20D and 23D).

As mentioned before, the major difference between measurement cartridges 10g and 10h is that cartridge 10g comprises a calibration fluid blister 75g for performing a one-point calibration. An option in cartridge 10g is inclusion of a directional valve element 67g (see FIGS. 20B, 20E, 20G and 21H). The smaller section 267g of directional valve element 67g closes off fluid communication with blood conduit 402g by folding against valve seat 333g (see FIG. 21H), when calibration liquid from ruptured blister 75g is forced, through conduits 431g, 433g, 403g and 261g in that order, preventing mixing of blood and calibration liquid. Subsequently after the calibration liquid is used to perform a one-point calibration of sensors in electrochemical sensor array 61g, pressurized air from air bladder 417g pushes the blood from blood conduit 402g into the electrochemical sensor chamber 261g for blood measurement, and the pressure from the blood sample pushes the smaller section 268g of directional valve element 68g against the outlet of conduit 433g, preventing the blood from flowing towards the blister 75g.

### Spectroscopic Measurement

Spectroscopic measurement of a blood sample is described. Other terms like spectrophotometric, photometric or optical measurement are sometimes used instead of spectroscopic measurement. A block diagram of an example of a system 70 (lower panel) for measuring one or more analyte quantities per unit volume of blood and one or more formed element quantities per unit volume of blood is provided as a non-limiting example in FIG. 15. Output displays of the analyzer are an image of cells in blood (upper left panel) and an absorbance spectrum (upper right panel). For spectroscopic measurement alone, one can replace the beam splitter of analyzer 70 (bifurcated optical fiber 16 shown as an example) with a straight optical fiber connected directly to an EMR (electromagnetic radiation) dispersing element 28 (a grating shown as a non-limiting example), and eliminate elements 18 (magnifying system of analyzer 70), 22 (two-dimensional multi-channel detector of analyzer 70), 24 (analog to digital converter of analyzer 70), and 26 (processor of analyzer 70).

With respect to the spectroscopic measurement alone, the analyzer may comprise a source of EMR (represented by 12 in FIG. 15) for interrogating the sample and measuring the EMR transmitted through the sample in the optical chamber of a generic measurement cartridge 10, fully inserted in a receptor 14 of the analyzer of system 70. A spectrometer of the system 70 comprises a one-dimensional multi-channel detector 32 arranged as a linear PDA (photo diode array) detector, for example, a linear repetitive installation of discrete photodiodes (may be referred to as pixels) on an integrated circuit chip. For measuring transmittance, the source of EMR and the PDA detector should be on opposite sides of the optical chamber, and for measuring reflectance, both the source of EMR and the PDA detector should be on the same side of the optical chamber. For reflectance measurement, the distal optical window of the optical chamber may be used as a reflecting member. Alternatively, a reflecting member may be installed in the cartridge receptor of the analyzer, and in close proximity to the optical window distal to the source of EMR. In other examples, EMR reflected from the sample may be measured. The source of EMR, which impinges upon, illuminates or interrogates the contents of the optical chamber, may be a tungsten lamp (other lamps may be used), one or more lasers, and one or more light-emitting diodes (LEDs) across a range of wavelengths as is well known in the art, and without being limited in any way. The analyzer may also include a spectrometer, which may comprise multichannel detectors such as a photodiode array (PDA), a charge-coupled device (CCD), or a complementary metal oxide semiconductor (CMOS), for example, without being limited in any way. The spectrometer may also comprise a prism, a transmission grating or a reflecting (or reflection) grating for dispersing EMR reflected from a sample (i.e., reflectance, denoted by R) or EMR transmitted through a sample (i.e. transmittance, denoted by T), into component wavelengths.

For illustration of a method for performing spectroscopic measurement of whole blood, and by way of example which is not to be considered limiting, the PDA detector may have a pixel dispersion of 2 nanometers per pixel (i.e., the pixel or digital resolution), and the PDA detector is calibrated (i.e., wavelength calibration) to read from wavelengths 300 nanometers to 812 nanometers. Two laser beams may be used to conduct wavelength calibration, which is well known by persons having knowledge in the art (see for example U.S. Pat. Nos. 6,372,503, and 6,711,516). In this example, the center of pixel 1 is assigned a wavelength of 300 nanometers (laser #1), and the center of pixel 256 is assigned a wavelength of 812 nanometers (laser #2), thereby providing a wavelength range of 300-812 nanometers. For clarity, since the center of pixel 1 is assigned 300 nanometers, the center of pixel 2 will be assigned 302 nanometers, the center of pixel 3 will be assigned 304 nanometers and so on in increments of 2 nanometers per pixel (the pixel dispersion). The two lasers may emit EMR at any wavelength within the range of 300-812 nanometers, having sufficient spacing so that linear interpolation and linear extrapolation of wavelengths can be conducted. A person skilled in spectroscopy should appreciate that the wavelength range and spectral resolution of the PDA detector depends on several factors, for example, the semiconductor material used to construct the PDA, and diffraction grating (transmission or reflective/reflection grating) and the orientation of the grating relative to the PDA detector. The source of EMR is a major determinant of the wavelength range. Each pixel is typically scanned in microseconds, which provides sufficient time to accumulate sufficient charge on the photodiode, for example to distinguish a signal from noise and dark current, without saturating the photodiode. The time the photodiode is exposed to the EMR may be referred to as “integration time”.

Saturation, or “saturating the photodiode”, means that the photodiode has reached a maximum response in current and any additional photons impinging upon the photodiode is usually converted to heat instead of current. Because the scanning time is so short, it is reasonable to say that all the photodiodes in the PDA detector are scanned simultaneously. The photons are converted to electrical current, which is measured and digitized. In this present example, absorbance (sometimes referred to as absorption, denoted by A) may be determined, where

$$A = - {log}_{10} T .$$

It is well known that transmittance is defined as the fraction of incident light which is transmitted or passes through a sample. Thus:

$$T = I / {I}_{0} ,$$

where

Iₒ=the intensity of light (or EMR) impinging upon or interrogating the sample (i.e. the incident light) and

I=the intensity of light (or EMR) emerging from the sample after passing through the sample.

For calculating transmittance, the amount of EMR impinging upon the optical chamber, Iₒ, may be measured by interrogating an optical chamber containing air. The EMR impinging upon the optical chamber, Iₒ, may be measured before or after every sample measurement, or less frequently and stored in the processor for later use.

As an example, spectroscopic measurements are used to estimate prothrombin time (PT; usually reported as PT-INR; PT-International Normalized Ratio), activated partial thromboplastin time (aPTT), or thrombin time (TT), and since a normal PT is about 10-14 seconds, a normal ACT is about 70-130 seconds, and a normal TT is about 15-19 seconds, the measurements are performed every second. An aspect of the invention with respect to coagulation measurements, e.g. PT, ACT and TT, is to use the absorbance at one or more wavelengths or pattern recognition using absorbances at a plurality of wavelengths. Techniques of pattern recognition, combined with spectroscopy are known by those having skill in the art. An example where spectroscopy, combined with pattern recognition algorithms are used and that may be applied to the methods described herein, is provided in Zhang et. al. (Mid-Infrared Spectroscopy for Coffee Variety Identification: Comparison of Pattern Recognition Methods”, J. of Spectroscopy, Volume 2016, Article ID 7927286). As blood coagulates, the blood changes from various liquid varieties to various gel varieties, with corresponding changes in spectroscopic patterns, allowing one to use similar techniques as those used by Zhang et. al. to identify different variety of coffee beans. The specific blood coagulation time measured depends on the reagents included in the cartridge. For example, thromboplastin may be used for PT, celite or kaolin may be used for ACT, and thrombin may be used for TT.

Typically, blood coagulation time is measured using mechanical methods. For spectroscopic-based assays, citrated plasma is usually used in place of whole blood, because with whole blood, a much larger fraction of the incident EMR is scattered and absorbed by the blood cells, compared with the change in emerging EMR due to gelling of the plasma. However, separating out the plasma from the whole blood requires time and centrifugation equipment. It is well known that as plasma clots or coagulates, the absorbance at a single wavelength increases. By way of example, G. O. Gogstad et. al. (1986, “Turbidimetric Determination of Prothrombin Time by Clotting in a Centrifugal Analyzer” Clin. Chem. 32/10, 1857-1862), describe the change in absorbance spectra of plasma during coagulation. However, measurement of coagulation time using whole blood instead of plasma is more representative of in vivo coagulation. Therefore, there is a need for spectroscopic measurement of the blood coagulation time employing whole blood. In order to improve the signal to noise ratio when whole blood is used with the devices as described herein, the depth of the optical chamber should be relatively small, for example about 50-200 micrometers. The use of absorbance, reflectance or transmittance at a single wavelength to generate a clotting reaction curve (for example as shown in FIG. 1 of Gogstad et. al. 1986, using absorbance), and the calculations used to compute clotting time, are considered to be within the scope of the present invention. Gogstad et. al. also provided examples of calculations use to compute clotting time that may be used according to the methods described herein.

As an example, the source of EMR may be a tungsten lamp. U.S. Pat. No. 6,651,015 describes how spectrophotometric apparatus are calibrated for measuring properties of blood, using multi-wavelength analysis. With the use of a source of EMR like a tungsten lamp, which provides multiwavelength EMR (the tungsten lamp is polychromatic, whereas a laser is monochromatic), and the use of a linear PDA detector, the analyzer has the capacity to generate full absorbance spectra in milliseconds. Several spectra may be collected over milliseconds and the absorbances averaged to minimize noise. Mathematical smoothing techniques, which are covered extensively in the literature, may be used to minimize noise. Other mathematical techniques like the use of an order derivative of absorbance are also discussed in U.S. Pat. No. 6,651,015. Even though full absorbance spectra are obtained, selected portions of the absorbance spectra, a wavelength range of the absorbance spectra, or the full absorbance spectra, may be used in order to determine a concentration of one or more than one analyte of interest. Examples of an absorbance spectrum is provided in FIG. 15 (see 39).

Any analyte that provides an absorbance, reflection or transmission spectrum change at one or more wavelengths with a change in the concentration of the analyte may be measured by spectroscopy. Other examples of analytes include bilirubin and CO-oximetry.

### Electrochemical Measurement

Electrochemical measurements are performed using electrochemical sensors installed in the detection chamber of the measurement cartridge. The electrochemical sensors may contain, without being limiting in any way, at least one of an amperometric sensor (e.g. a glucose sensor comprising an enzyme glucose oxidase or a sensor that measures pO₂), a conductivity sensor (e.g. a hematocrit sensor or an electrical switch), and a potentiometric sensor (e.g. an ion-selective electrode that can measure an electrolyte or pH).

As an example, electrochemical sensor array 61b of measurement cartridge 10b, illustrated collectively in FIGS. 9A-9G. The electrochemical sensor array 61b comprises at least one of an amperometric sensor, a conductivity sensor and a potentiometric sensor, and is disposed in a biosensor chamber 261b along a blood flow path. Some electrochemical sensors comprise at least one active surface exposed to the blood sample. Those skilled in the art will appreciate that biosensors may include various transducer arrangements that convert at least one property of the blood sample into an electrical signal. The electrical signal may be for example, a current, a voltage or a resistance/conductance. The transducer comprises at least one active surface for contacting the blood sample and the at least one active surface is one of a chemical sensitive surface, or an ionic sensitive surface, and wherein the at least one biosensor comprises at least one of a transistor, an ion-selective membrane, a membrane-bound enzyme, a membrane-bound antigen, a membrane-bound antibody, or a membrane-bound strand of nucleic acid. The cartridge 10b also comprises at least one electrical output contact, and the cartridge slot of the analyzer also comprises at least one electrical input contact, wherein the electrical output contact mates with the electrical input contact after the disposable cartridge is properly inserted into the receptor 14 of analyzer 80 illustrated in FIG. 18C. The electrochemical sensor array 61b is usually in a dry form, and is hydrated by the blood sample when the blood sample is allowed to flow over the electrochemical sensors. In some measurement cartridges, for example measurement cartridge 10g, illustrated collectively in FIGS. 19A-21J, the electrochemical sensor array 61g is hydrated by calibration liquid from blister 75g, prior to flow of blood over the electrochemical sensor array 61g. The calibration liquid in blister 76g is used to perform a one-point calibration (offset correction) of at least one of the sensors of electrochemical sensor array 61g. In addition, at infrequent intervals, calibration cartridge 20b may be used to perform a two-point calibration (i.e., offset and slope correction) electrochemical sensor array 61g.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein. Furthermore, the discussed combination of features might not be absolutely necessary for the inventive solution.

I claim:

1. A system for measuring one or more properties of a blood sample, the system comprising:
a measurement cartridge for measuring the one or more properties of the blood sample, the measurement cartridge comprising:
a measurement cartridge body having an upper surface and a lower surface, the upper surface defining a sample storage well for receiving the blood sample;
a measurement electrochemical sensor chamber located within the measurement cartridge body, the measurement electrochemical sensor chamber comprising at least one first electrochemical sensor for generating measurement electrical signals in response to the one or more properties of the blood sample; and
a blood flow conduit for establishing fluid communication between the sample storage well and the measurement electrochemical sensor chamber;
a calibration cartridge comprising:
a calibration cartridge body having an upper surface and a lower surface;
at least one sealed blister within the calibration cartridge body containing calibration liquid comprising known amounts of the one or more properties; and
a calibration electrochemical sensor chamber located within the calibration cartridge body, the calibration electrochemical sensor chamber comprising at least one second electrochemical sensor for generating calibration electrical signals in response to the calibration liquid, wherein the at least one first and second electrochemical sensors generate similar electrical signals in response to the same amount of the same one or more properties; and
a calibration liquid conduit for establishing fluid communication between the at least one sealed blister and the calibration electrochemical sensor chamber;
and
an analyzer comprising:
a receptor for separately receiving the calibration cartridge and the measurement cartridge;
means for releasing calibration liquid from the at least one sealed blister containing the calibration liquid;
means for moving the calibration liquid from the at least one sealed blister to the at least one second electrochemical sensor of the calibration cartridge;
means for moving the blood to the at least one first electrochemical sensor of the measurement cartridge;
an electrical receiver for receiving the calibration electrical signals generated by the at least one second electrochemical sensor and for receiving the measurement electrical signals generated by the least one first electrochemical sensor; and
a processor for developing a mathematical relation between the calibration electrical signals and the one or more properties in the calibration liquid, and applying the mathematical relation to the measurement electrical signals to determine the amount of the one or more properties in the blood sample.

2. The system of claim 1, wherein the at least one sealed blister consists of one sealed blister containing calibration liquid, for performing one-point calibration of the at least one first electrochemical sensor.

3. The system of claim 1, wherein the at least one sealed blister consists of two sealed blisters containing calibration liquid, for performing two-point calibration of the at least one first electrochemical sensor.

4. The system of claim 1, wherein the system comprises a plurality of calibration cartridges, for performing multi-point calibration, and wherein each of the plurality of calibration cartridges comprises a single calibration liquid blister in order to provide a plurality of calibration liquid blisters, and wherein each of the plurality of calibration liquid blisters comprises a different liquid composition.

5. The system of claim 1, wherein the one or more properties of the blood sample is pH and the at least one first electrochemical sensor and the at least one second electrochemical sensor are potentiometric electrochemical sensors.

6. The system of claim 1, wherein the measurement cartridge further comprises an optical chamber having at least one of an upper optical window and a lower optical window, the optical chamber in fluid communication with the blood flow conduit, the optical chamber for facilitating interrogation of a portion of the blood sample by electromagnetic radiation, for measuring one or more other properties of the blood.

7. The system of claim 1, wherein the means for moving the blood sample to the at least one first electrochemical sensor of the measurement cartridge comprises at least one of:
an air bladder disposed in the measurement cartridge body, the air bladder in fluid communication with the sample storage well;
an analyzer pump attachable to a duct of the measurement cartridge body and in fluid communication with the sample storage well;
a surface of the blood flow conduit sufficiently hydrophilic to promote blood flow by capillary action;
a cap for covering the sample storage well; and
at least one vent defined by a surface in the cartridge body or the cap in communication with the blood flow conduit.

8. The system of claim 1, wherein the measurement cartridge further comprises one or more reagents and means for mixing the blood sample and the one or more reagents.

9. The system of claim 1, wherein the sample storage well comprises a top portion for receiving the blood sample and a bottom portion for releasing at least a portion of the blood sample to the blood flow conduit, and wherein the measurement cartridge further comprises means for mitigating blood flow out of the bottom portion of the sample storage well when blood is received in the sample storage well through the top portion.

10. The system of claim 1, wherein the measurement cartridge further comprises a cap, the cap selected from a hinged cap, a pivotal cap, a sliding cap, and a screw cap for covering the sample storage well.

11. The system of claim 1, wherein the at least one first electrochemical sensor and the at least one second electrochemical sensor are of the same type manufactured in the same batch.

12. A calibration cartridge for calibrating at least one electrochemical sensor used for measuring one or more properties of a blood sample, the calibration cartridge comprising:
a calibration cartridge body having an upper surface and a lower surface;
at least one sealed blister located within the calibration body and containing a calibration liquid, wherein the calibration liquid comprises a known amount of the one or more properties of the blood sample;
means for releasing the calibration liquid from the at least one sealed blister;
a first calibration liquid conduit in fluid communication with each of the at least one sealed blister for receiving the calibration liquid;
a second calibration liquid conduit for receiving calibration liquid from each first calibration liquid conduit, wherein the second calibration conduit is closed off from any other liquid influx;
an electrochemical sensor chamber in fluid communication with the second calibration liquid conduit, the electrochemical sensor chamber comprising at least one electrochemical sensor and at least one electrical output, when installed with an associated analyzer, the at least one electrical output is configured to make contact with at least one electrical input of the associated analyzer, used to measure the one or more properties of the blood sample; and
a vent in communication with the electrochemical sensor chamber, wherein the vent is for releasing pressure and allowing the calibration liquid to make contact with the at least one electrochemical sensor.

13. The calibration cartridge of claim 12, wherein the calibration cartridge body does not include a sample storage well.

14. The calibration cartridge of claim 12, comprising one sealed blister containing calibration liquid, for performing one-point calibration of the at least one electrochemical sensor.

15. The calibration cartridge of claim 12, comprising two sealed blisters containing different calibration liquids, two first calibration liquid conduits, and one second calibration liquid conduit, for performing two-point calibration of the at least one electrochemical sensor.

16. The calibration cartridge of claim 15, further comprising a directional valve disposed at the junction of the two first calibration liquid conduits and the second calibration liquid conduit.

17. The calibration cartridge of claim 12, wherein the means for releasing calibration liquid comprise: (a) at least one spike for rupturing the at least one sealed blister; or (b) a weakened portion of each of the at least one sealed blister for rupturing the at least one sealed blister, wherein when the calibration cartridge is installed with an associated analyzer, a force on the at least one sealed blister is provided by the associated analyzer.
